(19) United States
(12) Patent Application Publication
(10) Pub. No.: US 2023/0287050 A1
(43) Pub. Date: Sep. 14, 2023

(54) Immunogenic Compounds

(71) Applicant: AC Immune SA, Lausanne (CH)

(72) Inventors: Dorian Winter, Wien (AT); Günther Staffler, Wien (AT); Gergana Galabova, Wien (AT); Eva Mihailovska, Wien (AT); Petra Gruber, Wien (AT); Katja Balazs, Wien (AT)

(21) Appl. No.: 18/014,846

(22) PCT Filed: Aug. 4, 2021
(86) PCT No.: PCT/EP2021/071778

§ 371 Date: Jan. 6, 2023

(30) Foreign Application Priority Data
Aug. 4, 2020 (EP) 20189425.0

(51) Int. Cl. C07K 7/08; A61P 25/28; A61K 47/66; A61K 39/00

(52) U.S. Cl. CPC C07K 7/08; A61P 25/28; A61K 47/66; A61K 39/0007; A61K 38/00

(57) ABSTRACT

An antigenic peptide comprises the structure X₁-X₂-X₃-X₄-X₅-X₆-X₇-P-X₉-X₁₀-X₁₁-X₁₂ and is derived from amino acids 113-124 of alpha synuclein. Ac-cording to the structure: P is proline; X₁ is L, K, A or S, wherein L is leucine, K is lysine, A is alanine and S is serine; X₂ is E or S, wherein E and S are as defined above; X₃ is D, E, K, N, A or S, wherein N is asparagine, D is aspartic acid and D, E, K, A and S are as defined above; X₄ is M, A, S, L or K, wherein M is methionine and A, S, L and K are as defined above; X₅ is P or A as defined above; X₆ is V, A or S, wherein V is valine and A and S are as defined above; X₇ is D or S as defined above; X₉ is D or A as defined above; X₁₀ is N, S or A, wherein N, S and A are as defined above; X₁₁ is E, A or S, wherein E, A and S are as defined above; X₁₂ is present or not and, if present, is A, K, V, S, or G wherein G is glycine and A, K, V and S are as defined above. The structure comprises at least one mutation compared to the wild type L-E-D-M-P-V-D-P-D-N-E-A sequence. The peptide does not comprise the dipeptide Y-E immediately following X12, wherein Y is tyrosine and E is as defined above. The peptides are conjugated to a suitable carrier and useful in treating synucleinopathies.

The invention relates to immunogenic compounds and their use in the prevention and treatment of synucleopathies, especially of Parkinson's disease (PD), Dementia with Lewy bodies (DLB) and Multiple System Atrophy (MSA).

PD is a synucleinopathy and the second most common neurodegenerative movement disease. PD prevalence ranges between 100 and 200/100,000 in the general population, and affects approximately 1% of the population above the age of 60 with an annual incidence of about 15/100,000. It is a chronic progressive disorder, defined by a combination of motoric syndromes (bradykinesia, rigidity, resting tremor and postural instability) and by non-motoric syndromes (a variety of autonomic dysfunctions, sensory abnormalities, and psychiatric abnormalities) that usually precede motoric syndromes. The hallmark of the disease is a profound loss of dopaminergic neurons in the substantia nigra (SN), accompanied by the accumulation of filamentous protein inclusions, termed Lewy Bodies (LB), which are predominantly composed of alpha-synuclein (aSyn). PD, DLB and other LB diseases show accumulation and re-distribution of aSyn in various brains regions and cellular populations.

MSA is another very important synucleinopathy. MSA is a sporadic neurodegenerative disorder that is characterised by symptoms of L-DOPA-resistant parkinsonism, cerebellar ataxia, and dysautonomia. Patients suffer from multisystem neuronal loss affecting various brain areas including striatum, substantia nigra, cerebellum, pons, as well as the inferior olives and the spinal cord. MSA is characterized by aSyn-positive glial cytoplasmic (GCI) and rare neuronal inclusions throughout the central nervous system. These inclusions are associated with striatonigral degeneration, olivopontocerebellar atrophy, and involvement of autonomic nuclei in medulla and spinal cord. The importance of GCIs for the pathogenesis of MSA is generally acknowledged and underscored by recent analysis of transgenic mouse models analysing the effect of aSyn overexpression in oligodendroglia. In tg mice overexpressing human aSyn both GCI-like aggregates and biochemical markers of MSA were observed.

DLB is the second most common type of neurodegenerative dementias in western society after Alzheimer's disease (AD). It makes up for 4-7% of clinically diagnosed dementia, with the same number of cases predicted to escape correct clinical diagnosis. Diagnosis of DLB is challenging, as the disease represents an “in-between” of AD and PD and shows overlapping features of both entities. The four clinical consensus criteria, of which two must be present to diagnose “probable DLB” are fluctuation in cognition and attention, recurrent visual hallucinations, REM sleep behaviour disorder and spontaneous parkinsonian motor signs, which occur later in the disease than the other criteria. These can be supported by a variety of additional clinical criteria that can, but need not occur, such as syncope and transient episodes of unresponsiveness, apathy, anxiety, depressions, psychotic episodes and neuroleptic sensitivity and many others. Symptoms are not uniform among patients.

DLB pathology is characterized by proteinaceous inclusions termed Lewy Bodies (LB), predominantly composed of alpha synuclein (aSyn) that has a role in the loss of function and structure of the neurons. However, in DLB, the LB are found distributed diffusely throughout the cortices, while in PD, they are found predominantly in the dopaminergic neurons of the Substantia Nigra. The LB in DLB are less well demarcated, less eosinophilic and less filamentous than those of PD. In addition, amyloid plaques containing mainly carboxy-terminally elongated forms of amyloid beta (Abeta) such as Abetal-42 can be found in the brains of DLB patients. Cortical amyloid deposition is associated with lower temporal lobe perfusion and a trend to hippocampal atrophy.

aSyn is a 14 kD naturally monomeric protein that is normally located to presynaptic terminals either bound to membranes of the synaptic vesicles or in the cytosol. Its natural function remains poorly understood and is likely involved in the synaptic transmission. During pathogenesis misfolding and aggregation of aSyn occurs in the central nervous system (CNS) and the peripheral nervous system, possibly as a consequence of posttranslational modification, including among others C-terminal protease cleavage (Dufty 2007, Bassil 2016). Aggregation leads to the generation of different aSyn species that have been associated with the pathogenesis of LB diseases, such as oligomers, protofibrils, and fibrils. The fibrillar forms of aSyn are detected mostly in LBs which are located in neuronal cell body (Kosaka et al., 1990, Dickson et al, 1989). Aggregates of aSyn can be also detected in astroglial cells (Braak 2007).

Not only fibrils, but various oligomeric aSyn species were detected in diseased human brains. In contrast to fibrillar aSyn, oligomeric aggregates are most likely located in neuronal projections and presynaptic terminals where they might damage the syn-apses, thus oligomeric aSyn has been attributed to cellular cytotoxicity.

It has been shown that monomeric aSyn can form different types of aggregates with different appearances, conformations, cytotoxicities and chemical properties under different in vitro conditions. Depending on the conformation of the monomer and the pre-vailing permissive conditions, different types of aggregates can develop, possessing different structural characteristics. When seeded, distinct aSyn-strains impress (e.g. “fibrils” or “ribbons”) their conformation upon the receiving cell and generate aggregates of the same strain in a process termed “conformational templating”. If they are injected into rat brains, these types of aggregates show varying properties in terms of inclusion formation and generation of behavioural and neurotoxic phenotypes in vivo. It is reasoned that different types of aSyn aggregates expose different polypeptide chains due to their distinct conformations. These differentially exposed surfaces would allow different sets of intramolecular interactions. Thus, the conformation of a given aSyn-strain dictates its properties such as their propensity for seeding or the predilection for certain cell types. Experimental data start to emerge that demonstrate the different properties of aSyn-strains extracted from PD and MSA material; Analysis of pathologic brain material from patients with PD or MSA demonstrated different properties of transmissible aSyn aggregates.

Currently available treatments exclusively target symptoms, however, therapies that are able to modify the underlying neurodegeneration are still under development. Herein an aSyn Specific Active ImmunoTherapy (SAIT) approach is presented that targets predominately the oligomeric and neurotoxic forms of aSyn and thus may interfere with disease progression in synucleinopathies.

Vaccination with PD01 and PD03, previously developed AFFITOPE® s targeting aSyn, has proven efficacy in various animal models of aSyn aggregation disorders, reducing aSyn pathology, preservation of neuroinflammation, as well as amelioration of behavior deficits (Mandler et al. 2014; WO 2009/103105 A1, WO 2011/020133 A1, WO 2017/076873 A1). These peptides turned out to be safe and well-tolerated vaccines which are able to induce target-specific antibodies in humans.

WO 2005/108423 A1 discloses peptides conferring environmental stress resistance derived i.a. from aSyn, beta-synuclein (bSyn), or gamma-synuclein (GSyn), which—if present in a fusion protein with a fusion partner protein—may provide decreased denaturation and/or increased solubility to this fusion partner protein. WO 2018/151821 A1 discloses antibodies to aSyn which are useful for diagnosing, treating and preventing neurodegenerative diseases. These antibodies are created by immunizing with native aSyn or the allelic variant A53T and should bind preferentially to preformed fibrils (“PFF”). WO 2005/013889 A2 discloses fragments of native aSyn which are useful for the treatment or prophylaxis of a LB disease (LBD) or to provide monoclonal antibodies for the treatment or prophylaxis of LBD.

It is an object of the present invention to provide a medicament for use in preventing and treating synucleinopathies on the basis of a vaccine. It is a further object to provide vaccination peptides suitable for human use.

Moreover, the invention may provide improved immunogenic peptides which are improved with respect to their immunogenicity, which induce higher amounts of aSyn-specific antibodies in the periphery, and which induce higher amounts of aSyn-specific antibodies in the brain. Moreover, it is also desirable to increase target binding of the induced antibodies due to oligoclonal antibody response (a “prolonged epitope”).

Therefore, the present invention provides an antigenic peptide comprising, consisting essentially of or consisting of the structure:

X₁-X₂-X₃-X₄-X₅-X₆-X₇-P-X₉-X₁₀-X₁₁-X₁₂,
Wherein:
P is proline;
X₁ is L, K, A or S, wherein L is leucine, K is lysine, A is alanine and S is serine;
X₂ is E or S, wherein E is glutamic acid and S is as defined above;
X₃ is D, E, K, N, A or S, wherein N is asparagine, D is aspartic acid and E, K, A and S are as defined above;
X₄ is M, A, S, L or K, wherein M is methionine and A, S, L and K are as defined above;
X₅ is P or A as defined above;
X₆ is V, A or S, wherein V is valine and A and S are as defined above;
X₇ is D or S as defined above;
X₉ is D or A as defined above;
X₁₀ is N, S or A, wherein N, S and A are as defined above;
X₁₁ is E, A or S, wherein E, A and S are as defined above;
X₁₂ is present or not and, if present, is A, K, V, S, or G wherein G is glycine and A, K, V and S are as defined above,
with the proviso that X₁-X₂-X₃-X₄-X₅-X₆-X₇-P-X₉-X₁₀-X₁₁-X₁₂ is not L-E-D-M-P-V-D-P-D-N-E-A and which comprises between 1 and 5 amino acid differences compared with the amino acid sequence L-E-D-M-P-V-D-P-D-N-E-A and wherein the peptide does not comprise the dipeptide Y-E immediately following X₁₂, wherein Y is tyrosine and E is as defined above. Thus, single letter amino acid code is generally used herein.

The antigenic peptide may comprise between 1 and 5 amino acid differences (i.e. 1, 2, 3, 4 or 5 differences) or between 1 and 4 amino acid differences compared with the amino acid sequence L-E-D-M-P-V-D-P-D-N-E-A. The differences are generally amino acid substitutions (according to the options set out for each position, with the exception that X₁₂ may be deleted). It is preferred that there are between 1 and 3 amino acid differences and most preferably 2 amino acid differences from the amino acid sequence L-E-D-M-P-V-D-P-D-N-E-A (which is the wild type alpha synuclein sequence from amino acids 113-124). These differences can be selected from any of amino acids X₁-X₁₂, except for X₈ which is proline. In some embodiments, the antigenic peptide comprises amino acid differences compared with the amino acid sequence L-E-D-M-P-V-D-P-D-N-E-A at one or more positions selected from X₁, X₃, X₄ and X₁₂. Preferably, the antigenic peptide comprises two amino acid differences compared with the amino acid sequence L-E-D-M-P-V-D-P-D-N-E-A at positions selected from X₁, X₃, X₄ and X₁₂.

The antigenic peptides of the invention retain their ability to generate aSyn-specific antibodies when employed as an immunogen. Moreover, the antigenic peptides of the invention are more immunogenic than the corresponding wild type aSyn peptide (comprising the 12-mer L-E-D-M-P-V-D-P-D-N-E-A, p9524 (SEQ ID NO: 4)) in terms of generating aSyn-specific antibodies, as demonstrated in the comparative experiments herein. They are also more immunogenic than other aSyn peptides from the C terminal region of aSyn, which were shown to be less immunogenic than p9524; see Tables 1 and 2 below (e.g. p4456 (SEQ ID NO: 1) and p4572 (SEQ ID NO: 2)). These properties can be tested by one skilled in the art following the teachings herein, typically by testing the peptide in the form of a conjugate of the type described in detail herein (e.g. the peptide conjugated to CRM197 as carrier protein). The peptides may be administered to a suitable experimental animal, in particular a mouse and a sample removed (e.g. blood) at a suitable time period following administration (again, see the examples for specific details). aSyn antibody titer determination may be performed for example by ELISA, as described herein.

The antigenic peptides of the invention typically do not comprise further alpha synuclein amino acid residues after X₁₂. In particular they do not comprise the dipeptide Y-E immediately following X₁₂. As described herein peptides including the amino acids Y₁₂₅ and E₁₂₆ are predicted by in silico analyses to bind with high affinity to different allelic variants of MHCI and thus be potential cytotoxic T cell epitopes (www.syfpeithi.de). The antigenic peptides may, however, comprise a limited number of further N terminal amino acid residues. Thus, the antigenic peptides may comprise, consist essentially of or consist of the structure

Xa-Xb-X₁-X₂-X₃-X₄-X₅-X₆-X₇-P-X₉-X₁₀-X₁₁-X₁₂,
Wherein:
Xa is present or not and, if present, is G, wherein G is glycine;
Xb is G, wherein G is as defined above; and
X₁-X₁₂ are as defined above.

The antigenic peptides of the invention are thus typically 11-20 amino acids in length, preferably 12-14 amino acids in length (i.e. 12, 13 or 14 amino acids in length). It is particularly preferred that the antigenic peptides are 12 or 14 amino acids in length. The antigenic peptides of the invention produce an antibody response in the absence of a T-cell response. Thus, the antigenic peptides of the invention themselves do not typically contain T-cell epitopes, in particular cytotoxic T-cell epitopes.

As described further herein, the antigenic peptides of the invention are typically employed in the form of immunogenic compounds in which they are conjugated to a carrier. In order to facilitate the process of conjugation, the antigenic peptides of the invention may further comprise an amino acid that acts as a conjugation site. Typically, this amino acid is a terminal amino acid and is preferably positioned at the N terminus. In preferred embodiments, the antigenic peptide further comprises a terminal cysteine residue, preferably an N-terminal cysteine residue.

In some embodiments, X₁ is L, S, or K, X₂ is E or S, X₃ is S, D, E, A, K or N, X₄ is M, X₁₀ is N, and/or X₁₂ is A, S, K or V, preferably wherein X₁ is L or K, X₂ is E, X₃ is S, D, E, K or A, X₄ is M, X₁₀ is N, and/or X₁₂ is A, S or K, especially wherein X₁ is L or K, X₃ is D, K or S and X₁₂ is A. In those embodiments, the other positions in the peptide are preferably wild type alpha synuclein amino acids.

In some embodiments, X₁ is A, S or K. In some embodiments, X₂ is S. In some embodiments, X₃ is A, S, E, K or N. In some embodiments, X₄ is A, S, L or K. In some embodiments, X₅ is A. In some embodiments, X₆ is A or S. In some embodiments, X₇ is S. In some embodiments, X₉ is A. In some embodiments, X₁₀ is A or S, preferably S. In some embodiments, X₁₁ is A or S. In some embodiments, X₁₂ is S, V, G or K. According to these embodiments, it is preferred that there is a maximum of one, two or three further mutations in the antigenic peptide compared with the native L-E-D-M-P-V-D-P-D-N-E-A sequence. In some embodiments there are no further mutations in the antigenic peptide compared with the native L-E-D-M-P-V-D-P-D-N-E-A sequence.

In some embodiments, the antigenic peptide is selected from the group consisting of AEDMPVDPDNEA, KESMPVDPDNEA, LESMPVDPDNEA, LESMPVDPDNES, SEDMPVDPDNEA, SEKMPVDPDNEA LEEMPVDPDNEA, SESMPVDPDNEA, LEDMPVDPDNES, LEAMPVDPDNEA, LEDMPVDPDNEK, LEDMPVDPDNEV, LEKMPVDPDNEK, LSDMPVDPDNEA, LEKMPVDPDNEA, LEKMPVDPDNES, LENMPVDPDNEA, KESMPVDPDNEK and KEDMPVDPDNEA, preferably SEDMPVDPDNEA, SEKMPVDPDNEA, LEEMPVDPDNEA, LEKMPVDPDNEK, LESMPVDPDNEA, LESMPVDPDNES, KESMPVDPDNEA, KEDMPVDPDNEA, LEKMPVDPDNES, LEKMPVDPDNEA and LESMPVDPDNES, especially LEKMPVDPDNEA, KESMPVDPDNEK, KESMPVDPDNEA and KEDMPVDPDNEA. In preferred embodiments, the antigenic peptide comprises, consists essentially of or consists of the amino acid sequence KESMPVDPDNEA, GKESMPVDPDNEA, GGKESMPVDPDNEA or CGGKESMPVDPDNEA.

The antigenic peptides of the invention are typically employed in the form of immunogenic compounds in which they are conjugated to a carrier. The carrier acts as a source of T-cell epitopes to improve the immune response to the immunogenic peptides. Thus, the invention further provides an immunogenic compound comprising an antigenic peptide of the invention and a carrier comprising T-cell epitopes attached to the antigenic peptide. There is provided an immunogenic compound having the structure

Carrier-X₁-X₂-X₃-X₄-P-V-D-P-D-X₁₀-E-X₁₂,
wherein
Carrier is a polypeptide carrier covalently coupled to X₁; preferably comprising a linker moiety which covalently links a carrier molecule to the peptide X₁-X₂-X₃-X₄-P-V-D-P-D-X₁₀-E-X₁₂;
D is aspartic acid, E is glutamic acid, P is proline, and V is valine;
X₁ is L, K, A or S, wherein L is leucine, K is lysine, A is alanine and S is serine;
X₂ is E or S, wherein E and S are as defined above;
X₃ is D, E, K, N, A or S, wherein N is asparagine and D, E, K, A and S are as defined above;
X₄ is M, A, S, L or K, wherein M is methionine and A, S, L and K are as defined above;
X₁₀ is N, S or A, wherein N, S and A are as defined above;
X₁₂ is present or not and, if present, is A, K, V, S, or G, wherein G is glycine and A, K, V and S are as defined above,
with the proviso that X₁-E-X₃-X₄-P-V-D-P-D-X₁₀-E-X₁₂ is not L-E-D-M-P-V-D-P-D-N-E-A.

The present compounds contain peptides which are able to elicit a strong anti-aSyn antibody response (“antigenic peptides”), i.e. that the induced antibodies show high cross-reactivity with human aSyn, although these peptides have a sequence which is different from the native sequence (L-E-D-M-P-V-D-P-D-N-E-A). Immune responses superior to the response with the native sequence (i.e. targeting the same native structures) was achieved with the peptides according to the present invention and a highly effective and suitable antibody response can be induced in vaccinated individuals. The antibodies induced by the vaccination with the compounds according to the present invention bind to the aggregated toxic aSyn species and Lewy bodies in pathological human brain tissue with high selectivity and specificity.

It was previously demonstrated that immunogenic peptides with non-native amino acid sequences of aSyn can be provided to elicit specific immune responses against aSyn which is improved with respect to cross-reactivity to bSyn (WO 2009/103105 A1, WO 2011/020133 A1). It has now been surprisingly demonstrated that improved peptides with non-native aSyn amino acid sequence can be produced which, in addition to eliciting an immune response specific to aSyn, demonstrate increased immunogenicity and are able to elicit antibodies with a higher cross-reactivity than the peptides described in WO 2009/103105 A1 and WO 2011/020133 A1 (see the Examples below).

Preferred immunogenic compounds according to the present invention comprise a preferred peptide of the present invention, wherein X₁ is L, S, or K, X₂ is E or S, X₃ is S, D, E, A, K or N, X₄ is M, X₁₀ is N, and/or X₁₂ is A, S, K or V. Other preferred immunogenic compounds according to the present invention comprise a preferred peptide, wherein X₁ is L, S or K, X₂ is E, X₃ is S, D, E, K or A, X₄ is M, X₁₀ is N, and/or X₁₂ is A, S or K. Another preferred embodiment is a peptide according to the present invention, wherein X₁ is L or K, X₃ is D, K or S and X₁₂ is A.

In preferred embodiments, the carrier comprising T-cell epitopes is attached to the antigenic peptide via a linker. Thus, there is also provided an antigenic peptide of the invention attached to a linker. Any suitable linker may be used, as would be readily appreciated by one skilled in the art. The linker may be a chemical linker or a peptide (amino acid based) linker. A linker may contain reactive functional groups to enable cross-linking of the antigenic peptide antigen to the carrier through a suitable chemical reaction. The linker may thus comprise two reactive groups. The first attaches to the carrier (protein), typically via a reactive amino acid side chain, such as through reaction with a primary amine (e.g. on a lysine residue). Thus, typically an amide bond is formed. The second attaches to the antigenic peptide, again typically via a reactive amino acid side chain, such as with a sulfhydryl group (e.g. on a cysteine residue). Thus, typically a thioether bond is formed. Preferred linkers are therefore heterobifunctional linkers, in particular those that contain an amine-reactive group, such as a N-hydroxysuccinimide (NHS) ester, and a sulfhydryl reactive group, such as a maleimide. Thus, the linker may comprise sGMBS (=sulfoGMBS) —(Maleimidobutyryloxy)sulfo succinimide ester, GMBS—Maleimidobutyryloxy succinimide ester, succinimidyl 3-(bromoacetamido) propionate (SBAP) or succinimidyl 6-(N-maleimido)-n-hexanoate (MHS). Other linkers that may be employed include sEMCS (=sulfoEMCS) —N-(ε-Maleimido-caproyloxy)sulfosuccinimide ester, MBS—m-Maleimidobenzoyl-N-hydroxysuccinimide ester, sMBS (=sulfoMBS) —(m-Maleimidobenzoyl-N-hydroxy)sulfo succinimide ester, iodo-acetamide-(PEG)2-maleimide (=N-(2-(2,5-dioxo-2,5-dihydro-1H-pyrrol-1-yl)ethyl)-3-(2-(2-iodoacetamido)ethoxy)propanamide), and iodo-acetamide-(PEG)-tri(maleimide) (=3,3′-((2-((3-((2-(2,5-dioxo-2,5-dihydro-1H-pyrrol-1-yl)ethyl)amino)-3-oxopropoxy)methyl)-2-(2-iodoacetamido)propane-1,3-diyl)bis(oxy))bis(N-(2-(2,5-dioxo-2,5-dihydro-1H-pyrrol-1-yl)ethyl)propanamide)). The linker may attach the antigenic peptide to specific amino acid residues, or side chains thereof, contained within the carrier (protein). Thus, in some embodiments, the antigenic peptide is conjugated to lysine residues (via the primary amine group) contained within the carrier (protein). Conjugation via histidine residues is also envisaged. It should be noted that the antigenic peptide may be provided in the context of a larger peptide molecule, the remainder of which is not derived from the alpha synuclein amino acid sequence in order to provide a linker, or to facilitate linkage. For example, the peptide can include additional residues, such as one or more cysteine residues with or without a spacer, such as polyethylene glycol (PEG), to facilitate attachment to the carrier (protein). Those additional residues are typically found at the N and/or C terminus of the antigenic peptide, preferably at the N terminus of the antigenic peptide. In this context, the term “consists essentially of” may mean that the antigenic peptide of the invention includes the 11-20, preferably 12 to 14 contiguous amino acids derived from the alpha synuclein sequence (amino acids 111-124 or 113-124, subject to at least one (and up to four) mutation as defined herein) but can include a limited number of additional residues, such as an additional cysteine residue, to facilitate attachment to the carrier protein with or without a spacer, such as PEG or an amino acid based spacer.

According to a preferred embodiment of the present invention, the linker moiety of the immunogenic compound comprises at least one cysteine and/or glycine amino acid residue, preferably coupled with a chemical linker to the polypeptide carrier moiety. Providing amino acid linkers at the N-terminal end of the (e.g. 12mer) peptides of the present invention offers many benefits for the coupling of larger compounds (as carriers) and for the elicitation of strong(er) immune responses; however, such linkers, especially amino acid linkers are not mandatory for the present invention.

Preferred amino acid linkers are glycine and cysteine (or combinations thereof, such as CG-, CGG-, CCG-, GC-, GGC-, GCC-, GG-, GGG-, etc.)) as well as isoleucine, alanine, valine, leucine, serine, glutamic acid, aspartic acid, lysine, asparagine, glutamine, etc. The amino acid linker may also consist of more than 1 amino acid residue, for example of 2, 3, 4, or 5 amino acid residues. It is preferential to exclude certain amino acid residues in the peptide (amino acid based) linker due to stability/folding issues, especially if the amino acid linker is longer than 5 amino acid residues and also if the antigenic peptide is directly coupled to a carrier protein via a peptide bond). Accordingly, if the amino acid linker (or the amino acid sequence extending N-terminally from X₁) is longer than 5 amino acid residues, it is preferred that the linker does not contain amino acids selected from the group consisting of proline, arginine, or histidine in the (linker) region N-terminal to X₁, i.e. within the first five amino acids of the amino acid linker molecule starting from the amino acid being N-terminal to X₁.

Preferred peptide linkers for use in the invention are those that do not form a T-cell epitope. This can be assessed using known methods, including in silico methods, such as by consulting the SYFPEITHI database of MHC ligands and peptide motifs (http://www.syfpeithi.de/).

Alternatively, or in addition to an amino acid linker, the Carrier preferably comprises—besides the polypeptide carrier also chemical linking groups (or chemical components resulting from chemical linking processes). Preferred linking groups may be obtained with the aid of chemical linkers, such as heterobifunctional compounds, such as GMBS or sulfo-GMBS. All chemical linkers known and used in the art, especially those which are used for producing products that are administered to human individuals may be used for providing linkage of the carrier to the peptide of the present invention. A specifically preferred chemical linker is a chemical linker which couples via a non-peptide bond to the antigenic peptide of the invention and to the carrier. Such non-peptide bond linking is specifically advantageous with respect to its immunogenic, stabilizing and/or manufacturing properties. Preferably, the linker moiety is formed by NHS-poly (ethylene oxide) (PEO) (e.g. by NHS-PEO₄-maleimide) or other compounds used in biochemical technology.

Specifically preferred immunogenic compounds according to the present invention comprise a preferred peptide X₁-X₂-X₃-X₄-P-V-D-P-D-X₁₀-E-X₁₂ which is selected from the group consisting of KESMPVDPDNEA, LESMPVDPDNEA, LESMPVDPDNES, SEDMPVDPDNEA, LEEMPVDPDNEA, SESMPVDPDNEA, LEDMPVDPDNES, LEAMPVDPDNEA, LEDMPVDPDNEK, LEDMPVDPDNEV, LEKMPVDPDNEK, LSDMPVDPDNEA, LEKMPVDPDNEA, KEDMPVDPDNEA, LENMPVDPDNEA, KESMPVDPDNEK and KEDMPVDPDNEA, preferably SEDMPVDPDNEA, LEEMPVDPDNEA, LESMPVDPDNEA, KESMPVDPDNEA, KEDMPVDPDNEA, LEKMPVDPDNEA and LESMPVDPDNES, especially LEKMPVDPDNEA, KESMPVDPDNEA and KEDMPVDPDNEA.

The carrier acts as a source of T-cell epitopes and thus typically comprises multiple T-cell epitopes. The T-cell epitopes are preferably universal T-cell epitopes. By “universal” T-cell epitope is meant an epitope that is specific to T-cells that are present in the majority of the human population. The “universal” ability of a T-cell epitope to activate T-cells is the result of at least two complementary properties: i) affinity of binding to the HLA groove, meaning the strength of the binding, as well as ii) its capacity to bind different HLA haplotypes in a promiscuous manner, meaning the ability to cover very diverse human populations, with regards to the differences in the expression of HLA molecules. The universal T-cell epitopes may bind to a majority of MHC class II alleles present in the human population. The T-cell epitopes of the carrier may thus be capable of stimulating a CD4 T-cell response. The T-cell epitopes of the carrier may thus be capable of stimulating a helper T-cell response that enhances (antigenic peptide specific) antibody production by B-cells.

According to a preferred embodiment, the immunogenic compound according to the present invention comprises a pharmaceutically acceptable polypeptide carrier molecule; a carrier protein. The carrier protein may be selected from the group consisting of diphtheria toxin (DT) and variants thereof, especially CRM197 (cross-reacting material 197), Keyhole Limpet Hemocyanin (KLH), tetanus toxoid, heat-labile enterotoxin (LT), cholera toxin (CT), tetanus toxoids (TT), mutant toxins, albumin-binding proteins, bovine serum albumin, and synthetically derived fusion peptides containing multiple T cell epitopes (e.g. Tet or PADRE). Further carrier protein that may be employed include pseudomonas exotoxin A (EPA), Haemophilus influenzae protein D (HiD) or meningococcal outer membrane protein complex (OMPC). CRM197 is a particularly preferred carrier protein.

Non-polypeptidic carriers may also be included in the immunogenic compounds of the present invention. Examples include poly(lactic-co-glycolic acid) microparticles (PLG microparticles), poloxamer particles, virus-like particles, and dendrimers. Further carriers may comprise nanoparticles or liposomes.

The immunogenic compounds and antigenic peptides of the present invention are preferably used for therapeutic and prophylactic methods for the treatment of human patients, especially for use in the treatment or prevention of synucleopathies. Preferred synucleopathies to be treated or prevented are Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I).

According to another aspect of the present invention, the present invention relates to a pharmaceutical preparation comprising an immunogenic compound or antigenic peptide according to the present invention and a pharmaceutically acceptable excipient (which may be referred to interchangeably as a carrier). The term “excipient” encompasses any component apart from the immunogenic compound that is present in the final formulation for administration. The pharmaceutical preparation is preferably for use as a vaccine in the treatment or prevention of a synucleinopathy, preferably a synucleinopathy selected from the group consisting of Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I).

The pharmaceutical preparation according to the present invention is preferably formulated as a vaccine. The pharmaceutical preparation, preferably a vaccine, may be formulated with an adjuvant, preferably with an adjuvant selected from the group consisting of MF59 aluminium phosphate, calcium phosphate, cytokines (e.g., IL-2, IL-12, GM-CSF), saponins (e.g., QS21), MDP derivatives, CpG oligos, IC31, LPS, monophosphoryl lipid A ((MPLA) which term encompasses MPLA-derivatives such as Monophosphoryl Hexa-acyl Lipid A, 3-Deacyl (Synthetic) (3D-(6-acyl) PHAD®), PHAD® (Phosphorylated HexaAcyl Disaccharide) or MPL), polyphosphazenes, and aluminium hydroxide, or mixtures thereof; especially with aluminium hydroxide as adjuvant. The purpose of the adjuvant(s) is to increase or stimulate the immune response in the subject. In some embodiments, the at least one adjuvant forms part of the carrier. Other adjuvants that may be employed according to the invention include aluminium containing adjuvants, in particular aluminium hydroxide (Alum), an imidazoquinolinamine such as Resiquimod (R-848) and/or CpG (synthetic oligodeoxynucleotides (ODNs) containing unmethylated CpG motifs) amongst others. The adjuvant may be a Toll-like receptor (TLR) agonist.

Typically, a pharmaceutical preparation according to the pre-sent invention, especially if formulated as a vaccine, contains the immunogenic compound according to the present invention (or the peptide of the present invention, optionally coupled to an alternative carrier) in an amount of from 0.1 ng to 10 mg, preferably 10 ng to 1 mg, in particular 1 μg to 500 μg, or, alternatively, e.g. 100 fmol to 10 μmol, preferably 10 pmol to 1 μmol, in particular 1 nmol to 500 nmol. The amount of the peptide may be 100 pmol to 100 nmol in some embodiments. The amounts herein refer to the peptide component of the composition.

Typically, the pharmaceutical preparation, especially the vaccine, may also contain auxiliary substances as excipients, e.g. buffers, stabilizers etc. Preferably, such auxiliary substances, e.g. a pharmaceutically acceptable excipient, such as water, buffer and/or stabilisers, are contained in an amount of 1 to 99% (weight), more preferred 5 to 80% (weight), especially 10 to 70% (weight). Preferably, the pharmaceutical preparation according to the present invention is formulated as liposomes, virosomes, iscoms, cochleates, emulsions.

The antigenic peptides, immunogenic compounds and pharmaceutical preparations of the invention may be administered according to any suitable schedule. They may be administered according to a prime-boost vaccination strategy. Prime-boost vaccination strategies involve multiple immunizations. They aim to improve the effectiveness of the vaccine. Generally, the same vaccine composition is administered each time; a so-called homologous prime-boost vaccination regimen. Possible administration regimes for the initial priming phase of the pharmaceutical preparation include a biweekly up to four—monthly treatment; 2 to 5, especially 3 to 4, initial priming vaccine administrations (in 1 to 5 months), followed by booster or maintenance vaccinations 3 to 12 months thereafter or even years thereafter are preferred—besides other regimes already suggested for other vaccines.

In absolute amounts, it is preferred to use an amount of the peptide (as antigen) of the present invention in the dose of at least 10 μg, preferably at least 50 μg. In this connection it is important to note that the “μg peptide (as antigen)” referred to in the present invention refers to the amount of antigenic peptide in the dose and does not include the carrier or linker part of the vaccine conjugate (immunogenic compound, if present). Accordingly, preferred amounts of antigen are at least 5 nmol, preferably at least 25 nmol.

Preferably, the pharmaceutical preparation comprising the immunogenic compound or the antigenic peptide is formulated for parenteral administration. In specific embodiments, the preparation is formulated for subcutaneous, intradermal or intramuscular administration. Intravenous administration may also be employed.

Vaccination strategy for the present invention preferably follows usual vaccination strategies. In a preferred embodiment, the vaccination strategy for self-antigens according to WO 2017/076873 A1 i.e. to elicit a primary immune response in a patient and then to perform on the patient booster or maintenance administrations.

Preferably, the booster/maintenance vaccination is administered at a point in time when the primary immune response has already passed, i.e. when the antibody titers elicited with a primary vaccination (elicited by one, two, three, four or more vaccine administration(s) within the course of the primary immune response elicitation) have dropped beyond certain levels (e.g. beyond a given threshold level of an assay suitable for testing high numbers of samples) or have at least gone under 30%, preferably under 50%, especially under 80%, of the maximum antibody level being present in the course of the primary vaccination, so as to obtain a high titer of antibodies over the whole treatment period. To keep such high levels, administration of booster/maintenance injections every 3 to 12 months after the initial (primary) immunization may be beneficial.

Preferably, the amount of antigen in the dose for the booster/maintenance administration is at least 20%, preferably at least 50%, more preferred at least 100%, especially at least 200%, higher than the amount used in the dose for the administration for the primary immune response. In certain embodiments, it is also preferred, if the amount of antigen in the dose for the booster/maintenance administration is at least 300%, preferably at least 400%, more preferred at least 500%, especially at least 600%, higher than the amount used in the dose for the administration for the primary immune response. However, in some embodiments, the same composition is administered on each occasion. Thus, in some embodiments the amount of antigen that is administered is the same on each occasion (within manufacturing tolerances).

According to a further preferred embodiment, the booster/maintenance administration is repeated after some time, for example after one, two, three, five or ten years. Preferably, the second or further booster/maintenance are performed in the same or similar manner than the first booster/maintenance administration, i.e. with the increased amount of antigen compared to the dose of the primary vaccination, or with the same amount of antigen.

Administration routes according to the present invention are usually the same routes as for current vaccinations. Therefore, preferred administration of the immunogenic compound or antigenic peptide, or pharmaceutical preparation, according to the present invention is parenteral, such as subcutaneous, intradermal or intramuscular administration. However, the immunogenic compound or antigenic peptide, or pharmaceutical preparation of the invention may be administered to the subject by any appropriate route of administration. As the skilled person would be aware, such compositions (preferably vaccine compositions) may be administered by topical, oral, rectal, nasal or parenteral (such as intravenous, intradermal, subcutaneous, or intramuscular) routes. In addition, the compositions may be incorporated into sustained release matrices such as biodegradable polymers, the polymers being implanted in the vicinity of, or in close proximity to, where de-livery is desired. However, in preferred embodiments, the compositions are administered intramuscularly or subcutaneously.

According to a preferred embodiment of the present invention, the immunogenic compound or antigenic peptide, or pharmaceutical preparation, preferably in the form of a vaccine, is administered together with an adjuvant, preferably aluminium oxyhydroxide. Ac-cording to this most preferred embodiment, the current invention relates to the use of European Pharmacopoeial grade (Aluminium-oxyhydroxide, monograph 1664), more specifically to the product manufactured by Brenntag Biosector (2% Alhydrogel) tested towards EP compliance. Alhydrogel is available in three varieties: Alhydrogel 1.3%; Alhydrogel 2% and Alhydrogel “85”. Alhydrogel 2% was elected as the International Standard Preparation for aluminium hydroxide gels. The pharmaceutical preparation according to the present invention is aseptically formulated into a suitable buffer, preferably an isotonic phosphate buffer (1 mM to 100 mM), preferably at a concentration of ≥1.0 mg/ml Alhydrogel (given as Al₂O₃ equivalent; this metric (Al as “Al₂O₃ equivalent”) is used generally for the present invention; accordingly, all doses and amounts referred to in the present application, as far they are relating to aluminium oxyhydroxide refer to Al₂O₃ equivalents (of aluminium oxyhydroxide (Alhydrogel)), even more preferably at a concentration of ≥1.5 mg/ml Alhydrogel (given as Al₂O₃ equivalent), most preferable at a concentration of ≥2.0 mg/ml Alhydrogel (given as Al₂O₃ equivalent). The amount of aluminium salt for Alhydrogel is given as Al₂O₃ equivalent in line with the strength as stated by the manufacturer (i.e. 2% Alhydrogel equates to 2% Al₂O₃, i.e. 20 mg/mL). This concentration is directly convertible into the respective concentration of aluminium by using the respective molecular masses (20 mg/mL Al₂O₃(Mw 101.96) corresponds to 10.6 mg/mL aluminium (molecular mass 26.98)).

The carrier of the present invention may be any suitable and pharmaceutically acceptable carrier moiety, optionally with a linker to couple the linker with the antigenic peptide of the invention (comprising X₁ to X₁₂).

According to a preferred embodiment of the present invention the antigenic peptide (which may be a 12mer peptide) of the present invention is coupled to a at least one pharmaceutically acceptable polypeptide carrier, preferably CRM197 (Cross reactive material 197), KLH (Keyhole Limpet Hemocyanin), tetanus toxoid, albumin-binding protein, bovine serum albumin, or synthetical fusion peptides containing multiple T cell epitopes. Further Carriers or carrier or linker moieties within the Carrier include a dendrimer (MAP; Biol. Chem. 358: 581), peptide linkers (or flanking regions) as well as the adjuvant substances described in Singh et al., Nat. Biotech. 17 (1999), 1075-1081 (in particular those in Table 1 of that document), and O'Hagan et al., Nature Reviews, Drug Discovery 2 (9) (2003), 727-735 (in particular the endogenous immuno-potentiating compounds and delivery systems described therein), and others or mixtures thereof. The conjugation chemistry (e.g. via heterobifunctional compounds such as GMBS and of course also others as described in “Bioconjugate Techniques”, Greg T. Hermanson) in this context can be selected from reactions known to the skilled person.

Moreover, a vaccine composition comprising an immunogenic compound or antigenic peptide according to the present invention may be formulated with an adjuvant, preferably a low soluble aluminium composition, in particular aluminium hydroxide. Of course, also adjuvants like MF59 aluminium phosphate, calcium phosphate, cytokines (e.g., IL-2, IL-12, GM-CSF), saponins (e.g., QS21), MDP derivatives, CpG oligos, IC31, LPS, MPLA (which includes MPL), polyphosphazenes, emulsions (e.g., Freund's, SAF), liposomes, virosomes, iscoms, cochleates, PLG microparticles, poloxamer particles, virus-like particles, heat-labile enterotoxin (LT), cholera toxin (CT), diphtheria toxin (DT), tetanus toxoids (TT), mutant toxins (e.g., LTK63 and LTR72), microparticles, liposomes and/or polymerized liposomes may be used.

The peptide or polypeptide of the present invention is preferably bound to the carrier or adjuvant via a linker, which is NHS-poly (ethylene oxide) (PEO) (e.g. NHS-PEO₄-maleimide). Alternatives are described above.

The carrier preferably comprises a toxoid protein. The toxoid protein may be a naturally occurring toxoid protein or a recombinant variant thereof used in pharmaceutical compositions. Toxins can be inactivated, for example, by treatment with formaldehyde, glutaraldehyde, UDP-dialdehyde, peroxide, oxygen or by mutation (e.g., using recombinant methods). Mutant diphtheria toxins with reduced toxicity can also be produced using recombinant methods.

DT is diphtheria toxin cross-reacting materials (DT-CRM) or diphtheria toxoids. An DT-CRM refers to a mutant diphtheria toxin, e.g., by mutation or by chemical modification, such that it no longer possesses sufficient ADP-ribosyl. Non-limiting examples of DT-CRM include DT-CRM30, DT-CRM45, DT-CRM176, DT-CRM197 and DT-CRM228. A diphtheria toxoid is a formaldehyde-inactivated diphtheria toxin. DT is commercially available from or can be prepared by methods known in the art, such as recombinant DNA technology.

CRM197 is a non-toxic variant (i.e., toxoid) of diphtheria toxin that retains the immunologic properties of the wild type diphtheria toxin. CRM197 differs from the wild type diphtheria toxin at a single base in the structural gene, which gives rise to a single amino acid substitution from glutamic acid to glycine. CRM197 is typically isolated from cultures of Corynebacterium diphtheria strain C7 (P1 97) grown on casamino acids and yeast extract-based medium. CRM197 may be purified through ultra-filtration, ammonium sulfate precipitation, and ion-exchange chromatography. Alternatively, CRM197 can be prepared recombinantly. CRM197 has been used in the design of glycoconjugate vaccines such as Hibtiter™, Menveo®, or Prevnar®.

Tetanus toxoid is prepared and used worldwide for large-scale immunization against tetanus (or lockjaw) caused by Clostridium tetani. Tetanus toxoid is also used both singly and in combination with diphtheria and/or pertussis vaccines. The parent protein, tetanus toxin, is generally obtained in cultures of Clostridium tetani. Tetanus toxin is a protein of about 150 kDa and consists of two subunits (about 100 kDa and about 50 kDa) linked by a sulfide bond. The toxin is typically detoxified with formaldehyde and can be purified from culture filtrates using known methods, such as ammonium sulfate precipitation, or chromatography techniques. Tetanus toxin may also be inactivated by recombinant genetic means. Tetanus toxoid has also been used as a carrier protein in other vaccines, including pneumococcal conjugate vaccines. Also mixed carrier can be used, e.g. pneumococcal conjugate vaccine in combination with CRM197, serotype 3 in combination with tetanus toxoid carrier, serotype 3 conjugated to diphtheria toxoid.

The peptides of the present invention are variants of the native human aSyn sequence, i.e. an AFFITOPE® or VARIOTOPE® (being a peptide that contains sequence variations compared with the original native aSyn sequence, but which shows similar (the same or improved) immunization characteristics, i.e. is able to elicit an immune response that is similar or higher than the immune response that is obtained with the native aSyn sequence). At the same time, the AFFITOPES® are designed not to elicit cytotoxic, or helper T cell responses, the first to avoid cytotoxic attacks against any tissue reachable by CD8⁺ T cells bearing linear sequence fragments of the immunizing peptide, the latter to avoid responses to target-derived peptides independently of the vaccine and thus the generation of a permanently renewed and uncontrolled immune response. This AFFITOPE® or VARIOTOPE® technology has been designed with the aim of (i) breaking tolerance against self-proteins, (ii) generating high titer antibody responses to the peptide moiety of the vaccine which cross-react with the native target protein epitope, (iii) and to not induce an autoimmune response.

In the course of the present invention, the length of the peptide has therefore been limited, preferably to 12 aa (X₁ to X₁₂). The antigenic peptides of the invention are typically 11-20 amino acids in length, preferably 12-14 amino acids in length. It is particularly preferred that the antigenic peptides are 12 or 14 amino acids in length. They typically contain between 1 and 4, preferably 2 or 3, amino acid mutations compared to the native alpha synuclein sequence. Note, however, that amino acid extensions may be permitted that are not based on (or identical to) the alpha synuclein sequence, especially at the N terminus of the peptide. Such additional amino acids may form part of a linker for example. Linkers (including amino acid, or peptide, linkers) may be present which covalently link the peptide to other molecular moieties, such as the Carrier. The linker may comprise additional amino acids, preferably amino acids with uncharged side chains such as glycines. The linkers may be attached to all positions in the immunogenic peptide, as long as the immunogenic properties of the peptides are not significantly worsened. For example, linkers may be linked via the N-terminal or C-terminal aa (X₁ or X₁₂); also linking inside the peptide may be possible. It is, however, preferred to link the present peptides via the N-terminus of the peptide (X₁) to the Carrier, if the peptides of the present invention are applied as immunogenic compositions. If the peptides of the present invention are used for other purposes, such as preparative moieties (e.g. in the course of antibody purification) or diagnostic probes (e.g. to detect antibodies in human specimen), linkers and carriers may be more diverse, including linking the peptides of the present invention to surfaces, i.e. solid surfaces.

Accordingly, the peptides of the present invention may also be used in various assays and kits, in particular in immunological assays and kits. Therefore, it is particularly preferred that the peptides of the present invention may be part of another peptide or polypeptide, for example, they may be fused or conjugated with an enzyme which is used as a reporter in immunological assays. Such reporter enzymes include e.g. fluorescent moieties, such as a green fluorescent protein (GFP), phosphatases, such as an alkaline phosphatase, or oxidases/reductases, such as a horseradish peroxidase.

In certain embodiments, the present invention relates to an antigenic peptide having the structure

X₁-X₂-X₃-X₄-P-V-D-P-D-X₁₀-E-X₁₂,
wherein
D is aspartic acid, E is glutamic acid, P is proline, and V is valine;
X₁ is L, K, A or S, wherein L is leucine, K is lysine, A is alanine and S is serine;
X₂ is E or S, wherein E and S are as defined above;
X₃ is D, E, K, N, A or S, wherein N is asparagine and D, E, K, A and S are as defined above;
X₄ is M, A, S, L or K, wherein M is methionine and A, S, L and K are as defined above;
X₁₀ is N, S or A, wherein N, S and A are as defined above;
X₁₂ is present or not and, if present, is A, K, V, S, or G wherein G is glycine and A, K, V and S are as defined above,
with the proviso that X₁-X₂-X₃-X₄-P-V-D-P-D-X₁₀-E-X₁₂ is not L-E-D-M-P-V-D-P-D-N-E-A.

In case of doubt, the term “having the structure” is to be understood as “consisting of (the structure)” the amino acid residues given (i.e. excluding further amino acid residues, e.g. at the C-terminus of the antigenic peptide). Minor modifications, such as amidation, esterification, formylation, acetylation, other chemical substitution etc. of a free C-terminal (or N-terminal) end of a peptide or its side chains are not excluded but it is preferred not to have such modifications present. Such minor modifications are within the scope of the term “consisting essentially of” as used herein. These 11mer or 12mer peptides according to the present invention (the “(antigenic) peptide(s) (of the present invention)”; “X₁ to X₁₂”; etc.) can be provided in compositions suitable for the intended use for preventing and/or treating synucleinopathies, especially in pharmaceutical compositions, preferably combined with a pharmaceutically acceptable carrier. Such pharmaceutical compositions can be administered to a patient in need thereof in an effective amount to achieve the preventive and/or therapeutic effect.

In one embodiment, the peptide according to the present invention is selected from the group consisting of AEDMPVDPDNEA, LEAMPVDPDNEA, LEDAPVDPDNEA, LEDMAVDPDNEA, LEDMPADPDNEA, LEDMPVDPANEA, LEDMPVDPDNAA, SEDMPVDPDNEA, LSDMPVDPDNEA, LESMPVDPDNEA, LEDSPVDPDNEA, LEDMPSDPDNEA, LEDMPVSPDNEA, LEDMPVDPDSEA, LEDMPVDPDNSA, LEDMPVDPDNES, LEEMPVDPDNEA, LEKMPVDPDNEA, LENMPVDPDNEA, LEDKPVDPDNEA, LEDMPVDPDNEV, LEDMPVDPDNEG, LEDMPVDPDNEK, LESMPVDPDNES, SESMPVDPDNEA, LESMPVDPDNEA, SEDMPVDPDNES, LEDSPVDPDNES, SEDSPVDPDNEA, KEDMPVDPDNEA, LEKMPVDPDNES, SEKMPVDPDNEA, LEKMPVDPDNEK, KESMPVDPDNEA, KESMPVDPDNEK, preferably, KEDMPVDPDNEA, LEKMPVDPDNEA, LESMPVDPDNES, LEKMPVDPDNES, SEKMPVDPDNEA, LEKMPVDPDNEK, KESMPVDPDNEA, KESMPVDPDNEK.

Preferably, the peptide according to the present invention is selected from the group consisting of KESMPVDPDNEA, LESMPVDPDNEA, LESMPVDPDNES, SEDMPVDPDNEA, LEEMPVDPDNEA, SESMPVDPDNEA, LEDMPVDPDNES, LEAMPVDPDNEA, LEDMPVDPDNEK, LEDMPVDPDNEV, LEKMPVDPDNEK, LSDMPVDPDNEA, LEKMPVDPDNEA, KEDMPVDPDNEA, LENMPVDPDNEA, KESMPVDPDNEK and KEDMPVDPDNEA, preferably SEDMPVDPDNEA, LEEMPVDPDNEA, LESMPVDPDNEA, KESMPVDPDNEA, KEDMPVDPDNEA, LEKMPVDPDNEA and LESMPVDPDNES, especially LEKMPVDPDNEA, KESMPVDPDNEA and KEDMPVDPDNEA.

According to another aspect, the present invention also refers to an antigenic peptide with an amino acid linker having the structure

Linker-X₁-X₂-X₃-X₄-P-V-D-P-D-X₁₀-E-X₁₂,
wherein X₁, X₂, X₃, X₄, X₁₀, and X₁₂ are defined as above, and wherein the amino acid linker comprises between 1 and 5 amino acid residues.

According to a preferred embodiment, the peptide with an amino acid linker according to the present invention comprises a linker, wherein the amino acid residue(s) in the linker is (are) selected from the group consisting of glycine, cysteine, isoleucine, alanine, valine, leucine, serine, glutamic acid, aspartic acid, lysine, asparagine, glutamine and combinations thereof, preferably wherein the linker is selected from the group C-, G-, CG-, CGG-, CCG-, GC-, GGC-, GCC-, GG-, and GGG-; especially wherein the peptide with an amino acid linker is selected from the group GGKESMPVDPDNEA, GKESMPVDPDNEA, GGGKESMPVDPDNEA, CGGKESMPVDPDNEA, GCGKESMPVDPDNEA, GGCKESMPVDPDNEA, CCGKESMPVDPDNEA, CCCKESMPVDPDNEA, CCCKESMPVDPDNEA, CCKESMPVDPDNEA, CGKESMPVDPDNEA, GCKESMPVDPDNEA, CKESMPVDPDNEA, GGKESMPVDPDNEK, GGKEDMPVDPDNEA GGKESMPVDPDNEK, GGKEDMPVDPDNEA, CLESMPVDPDNEA, CLESMPVDPDNES, CSEDMPVDPDNEA, CLEEMPVDPDNEA, CSESMPVDPDNEA, CLEDMPVDPDNES, CLEAMPVDPDNEA, CLEDMPVDPDNEK, CLEDMPVDPDNEV, CGGKESMPVDPDNEA, CLEKMPVDPDNEK, CLSDMPVDPDNEA, CLEKMPVDPDNEA, CKEDMPVDPDNEA, CLENMPVDPDNEA, CGGKESMPVDPDNEK, and CGGKEDMPVDPDNEA, especially GGKESMPVDPDNEA, GKESMPVDPDNEA, GGGKESMPVDPDNEA, CGGKESMPVDPDNEA, GCGKESMPVDPDNEA, GGCKESMPVDPDNEA, CCGKESMPVDPDNEA, CCCKESMPVDPDNEA, CCCKESMPVDPDNEA, CCKESMPVDPDNEA, CGKESMPVDPDNEA, GCKESMPVDPDNEA, and CKESMPVDPDNEA.

Preferably, according to all relevant aspects of the present invention
(a) X₁₂ is present and no further amino acid residue is present C-terminally to X₁₂; or
(b) X₁₂ is not present and no further amino acid residue is present C-terminally to X₁₁.

According to these specifically preferred embodiments the antigenic peptide of the invention has no further extensions at the C-terminal end (except that minor modifications may be present, e.g. which may stabilise the compound or peptide (e.g. amidation, etc.)). In any case, no amino acid extensions should (in contrast to the fusion proteins disclosed e.g. in WO 2005/108423 A1) be present C-terminally which represent the native amino acid sequence of aSyn, i.e. specifically Tyr₁₂₅ and Glu₁₂₆, which could bind with high affinity to different allelic variants of MHCI and thus could be potential cytotoxic T cell epitopes. Accordingly, no Y amino acid residue or YE dipeptide chain should be present at the C-terminal end of the peptide, wherein Y is tyrosine and E is as defined above.

The peptides and compounds of the present invention can be synthetically produced by chemical synthesis methods which are well known in the art, either as an isolated peptide or as a part of another polypeptide. Alternatively, the peptides and compounds can be produced in a microorganism which produces the peptide of the present invention which is then isolated and if desired, further purified. The peptides and compounds can be produced in microorganisms such as bacteria, yeast or fungi, in eukaryote cells such as a mammalian or an insect cell, or in a recombinant virus vector such as adenovirus, poxvirus, herpesvirus, Semliki forest virus, baculovirus, bacteriophage, Sindbis virus or Sendai virus. Suitable bacteria for producing the peptides and compounds include E. coli, B. subtilis or any other bacterium that is capable of expressing peptides such as the peptide mimotope. Suitable yeast types for expressing the peptides and compounds include Saccharomyces cerevisiae, Schizosaccharomyces pombe, Candida, Pichia pastoris or any other yeast capable of expressing peptides. Corresponding methods are well known in the art. Also methods for isolating and purifying recombinantly produced peptides and compounds are well known in the art and include e.g. gel filtration, affinity chromatography, ion exchange chromatography etc.

To facilitate isolation of the peptides and compounds, a fusion polypeptide may be made wherein the 12mer peptide is translationally fused (covalently linked) to a heterologous polypeptide which enables isolation by affinity chromatography. Typical heterologous polypeptides are His-Tag (e.g. His₆; 6 histidine residues), GST-Tag (Glutathione-S-transferase) etc. The fusion polypeptide facilitates not only the purification of the peptides and compounds but can also prevent degradation during purification. If it is desired to remove the heterologous polypeptide after purification the fusion polypeptide may comprise a cleavage site e.g. at the junction between the peptide mimotope and the heterologous polypeptide. The cleavage site consists of an amino acid sequence that is cleaved with an enzyme specific for the amino acid sequence at the site (e.g. proteases).

The antigenic peptide of the invention, the immunogenic compound of the invention or the pharmaceutical preparation of the invention are for use in therapy (i.e. as a medicament). More specifically, the invention provides the antigenic peptide of the invention, the immunogenic compound of the invention or the pharmaceutical preparation of the invention are for use in the treatment or prevention of a synucleinopathy. The invention also provides for use of the antigenic peptide of the invention, the immunogenic compound of the invention or the pharmaceutical preparation of the invention, for the manufacture of a medicament for the treatment or prevention of a synucleinopathy. The invention also provides a method for the treatment or prevention of a synucleinopathy comprising administering the antigenic peptide of the invention, the immunogenic compound of the invention or the pharmaceutical preparation of the invention to a subject in need thereof. In specific embodiments according to all of these aspects, the synucleinopathy is selected from the group consisting of Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I). In some embodiments according to these aspects, the term synucleinopathies (or alpha-synucleinopathies) is used to describe diseases where a-synuclein aggregates are detected and comprises primary synucleinopathies and concomitant pathology. Primary synucleinopathies include Parkinson's disease (sporadic, familial with alpha-synuclein mutations, familial with mutations other than alpha-synuclein, pure autonomic failure and Lewy body dysphagia), Lewy Body dementia (LBD; including dementia with Lewy bodies (DLB) (“pure” Lewy body dementia), Parkinson's disease dementia (PDD)), or Diffuse Lewy Body Disease, multiple system atrophy (Shy-Drager syndrome, striatonigral degeneration and olivopontocerebellar atrophy). Furthermore a-syn lesions can be detected as concomitant pathology in the following diseases: sporadic Alzheimer's disease, familial Alzheimer's disease with APP mutations, familial Alzheimer's disease with PS-1, PS-2 or other mutations, familial British dementia, inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

According to all these aspects, the subject that is treated is a mammalian subject, preferably a human. The antigenic peptide of the invention, the immunogenic compound of the invention or the pharmaceutical preparation of the invention is administered at an amount effective to treat or prevent a synucleinopathy. Prevention is preferred using vaccine compositions of the invention. Prevention encompasses delaying the onset and/or severity of disease (as compared to the situation without administration) as well as complete prevention of disease. Treatment encompasses ameliorating one or more symptoms of disease, preventing or delaying disease progression (as compared to the situation without administration) as well as complete treatment of disease. Suitable dosages and administration routes are described herein and variations may be determined empirically by a clinical practitioner.

The invention may be further defined by the following numbered clauses:

1. Immunogenic compound having the structure

Carrier-X₁-X₂-X₃-X₄-P-V-D-P-D-X₁₀-E-X₁₂,
wherein
Carrier is a polypeptide carrier covalently coupled to X₁; preferably comprising a linker moiety which covalently links a carrier molecule to the peptide X₁-X₂-X₃-X₄-P-V-D-P-D-X₁₀-E-X₁₂;
D is aspartic acid, E is glutamic acid, P is proline, and V is valine;
X₁ is L, K, A or S, wherein L is leucine, K is lysine, A is alanine and S is serine;
X₂ is E or S, wherein E and S are as defined above;
X₃ is D, E, K, N, A or S, wherein N is asparagine and D, E, K, A and S are as defined above;
X₄ is M, A, S, L or K, wherein M is methionine and A, S, L and K are as defined above;
X₁₀ is N, S or A, wherein N, S and A are as defined above;
X₁₂ is present or not and, if present, is A, K, V, S, or G, wherein G is glycine and A, K, V and S are as defined above,
with the proviso that X₁-X₂-X₃-X₄-P-V-D-P-D-X₁₀-E-X₁₂ is not L-E-D-M-P-V-D-P-D-N-E-A.
   2. Immunogenic compound according to clause 1, wherein X₁ is L, S, or K, X₂ is E or S, X₃ is S, D, E, A, K or N, X₄ is M, X₁₀ is and/or X₁₂ is A, S, K or V, preferably wherein X₁ is L or K, X₂ is E, X₃ is K or A, X₄ is M, X₁₀ is N, and/or X₁₂ is A, S or K, especially wherein X₁ is L or K, X₃ is D, K or S and X₁₂ is A.
   3. Immunogenic compound according to clause 1 or 2, wherein the linker moiety comprises at least one cysteine and/or glycine amino acid residue, preferably coupled with a chemical linker to the polypeptide carrier moiety, especially wherein the linker moiety is formed by NHS-poly (ethylene oxide) (PEO) (e.g. by NHS-PEO₄-maleimide).
   4. Immunogenic compound according to any one of clauses 1 to 3, wherein the peptide X₁-X₂-X₃-X₄-P-V-D-P-D-X₁₀-E-X₁₂ is selected from the group consisting of KESMPVDPDNEA, LESMPVDPDNEA, LESMPVDPDNES, SEDMPVDPDNEA, LEEMPVDPDNEA, SESMPVDPDNEA, LEDMPVDPDNES, LEAMPVDPDNEA, LEDMPVDPDNEK, LEDMPVDPDNEV, LEKMPVDPDNEK, LSDMPVDPDNEA, LEKMPVDPDNEA, LENMPVDPDNEA, KESMPVDPDNEK and KEDMPVDPDNEA, preferably SEDMPVDPDNEA, LEEMPVDPDNEA, LESMPVDPDNEA, KESMPVDPDNEA, KEDMPVDPDNEA, LEKMPVDPDNEA and LESMPVDPDNES, especially LEKMPVDPDNEA, KESMPVDPDNEA and KEDMPVDPDNEA.
   5. Immunogenic compound according to any one of clauses 1 to 4, wherein the peptide X₁-X₂-X₃-X₄-P-V-D-P-D-X₁₀-E-X₁₂ is KESMPVDPDNEA.
   6. Immunogenic compound according to any one of clauses 1 to 5, wherein the polypeptide carrier moiety is or comprises a pharmaceutically acceptable carrier molecule selected from the group consisting of Keyhole Limpet Hemocyanin (KLH), tetanus toxoid, heat-labile enterotoxin (LT), cholera toxin (CT), diphtheria toxin (DT) and variants thereof, especially CRM 197, tetanus toxoids (TT), mutant toxins, albumin-binding proteins, and bovine serum albumin.
   7. Immunogenic compound according to any one of clauses 1 to 6 for use in the treatment or prevention of synucleopathies, preferably for use in the treatment or prevention of a synucleinopathy selected from the group consisting of Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I).
   8. Pharmaceutical preparation comprising a compound according to any one of clauses 1 to 7 and a pharmaceutically acceptable carrier, preferably for use as a vaccine in the treatment or prevention of a synucleinopathy, preferably a synucleinopathy selected from the group consisting of Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I).
   9. Pharmaceutical preparation according to clause 8, wherein the preparation is formulated as a vaccine with an adjuvant, preferably with an adjuvant selected from the group consisting of MF59 aluminium phosphate, calcium phosphate, cytokines (e.g., IL-2, IL-12, GM-CSF), saponins (e.g., QS21), MDP derivatives, CpG oligos, IC31, LPS, MPL, polyphosphazenes, and aluminium hydroxide, or mixtures thereof; especially with aluminium hydroxide as adjuvant.
   10. Pharmaceutical preparation according to clause 8 or 9, wherein the compound according to any one of clauses 1 to 10 is contained in an amount from 0.1 ng to 10 mg, preferably 10 ng to 1 mg, in particular 100 ng to 100 μg.
   11. Pharmaceutical preparation according to any one of clauses 8 to 10, wherein the preparation is formulated for subcutaneous, intradermal or intramuscular administration; and/or wherein the preparation is formulated as liposomes, virosomes, iscoms, cochleates, or emulsions.
   12. Antigenic peptide having the structure

X₁-X₂-X₃-X₄-P-V-D-P-D-X₁₀-E-X₁₂
wherein
D is aspartic acid, E is glutamic acid, P is proline, and V is valine; (C-) is cysteine being present or not;
X₁ is L, K, A or S, wherein L is leucine, K is lysine, A is alanine and S is serine;
X₂ is E or S, wherein E and S are as defined above;
X₃ is D, E, K, N, A or S, wherein N is asparagine and D, E, K, A and S are as defined above;
X₄ is M, A, S, L or K, wherein M is methionine and A, S, L and K are as defined above;
X₁₀ is N, S or A, wherein N, S and A are as defined above;
X₁₂ is present or not and, if present, is A, K, V, S, or G wherein G is glycine and A, K, V and S are as defined above,
with the proviso that X₁-X₂-X₃-X₄-P-V-D-P-D-X₁₀-E-X₁₂ is not L-E-D-M-P-V-D-P-D-N-E-A.
   13. Peptide according to clause 12, selected from the group consisting of KESMPVDPDNEA, LESMPVDPDNEA, LESMPVDPDNES, SEDMPVDPDNEA, LEEMPVDPDNEA, SESMPVDPDNEA, LEDMPVDPDNES, LEAMPVDPDNEA, LEDMPVDPDNEK, LEDMPVDPDNEV, LEKMPVDPDNEK, LSDMPVDPDNEA, LEKMPVDPDNEA, LENMPVDPDNEA, KESMPVDPDNEK and KEDMPVDPDNEA, preferably SEDMPVDPDNEA, LEEMPVDPDNEA, LESMPVDPDNEA, KESMPVDPDNEA, KEDMPVDPDNEA, LEKMPVDPDNEA and LESMPVDPDNES, especially LEKMPVDPDNEA, KESMPVDPDNEA and KEDMPVDPDNEA.
   14. Peptide with an amino acid linker having the structure

Linker-X₁-X₂-X₃-X₄-P-V-D-P-D-X₁₀-E-X₁₂,
wherein X₁, X₂, X₃, X₄, X₁, and X₁₂ are defined as in clause 13, and
wherein the amino acid linker comprises between 1 and 5 amino acid residues.
   15. Peptide with an amino acid linker according to clause 14, wherein the amino acid residue(s) in the linker is (are) selected from the group consisting of glycine, cysteine, isoleucine, alanine, valine, leucine, serine, glutamic acid, aspartic acid, lysine, asparagine, glutamine and combinations thereof, preferably wherein the linker is selected from the group C-, G-, CG-, CGG-, CCG-, GC-, GGC-, GCC-, GG-, and GGG-; especially wherein the peptide with an amino acid linker is selected from the group GGKESMPVDPDNEA, GKESMPVDPDNEA, GGGKESMPVDPDNEA, CGGKESMPVDPDNEA, GCGKESMPVDPDNEA, GGCKESMPVDPDNEA, CCGKESMPVDPDNEA, CCCKESMPVDPDNEA, CCCKESMPVDPDNEA, CCKESMPVDPDNEA, CGKESMPVDPDNEA, GCKESMPVDPDNEA, CKESMPVDPDNEA, GGKESMPVDPDNEK, GGKEDMPVDPDNEA GGKESMPVDPDNEK, CLESMPVDPDNEA, CLESMPVDPDNES, CSEDMPVDPDNEA, CLEEMPVDPDNEA, CSESMPVDPDNEA, CLEDMPVDPDNES, CLEAMPVDPDNEA, CLEDMPVDPDNEK, CLEDMPVDPDNEV, CGGKESMPVDPDNEA, CLEKMPVDPDNEK, CLSDMPVDPDNEA, CLEKMPVDPDNEA, CKEDMPVDPDNEA, CLENMPVDPDNEA, CGGKESMPVDPDNEK, and CGGKEDMPVDPDNEA, especially GGKESMPVDPDNEA, GKESMPVDPDNEA, GGGKESMPVDPDNEA, CGGKESMPVDPDNEA, GCGKESMPVDPDNEA, GGCKESMPVDPDNEA, CCGKESMPVDPDNEA, CCCKESMPVDPDNEA, CCCKESMPVDPDNEA, CCKESMPVDPDNEA, CGKESMPVDPDNEA, GCKESMPVDPDNEA, and CKESMPVDPDNEA.
   16. Immunogenic compound according to any one of clauses 1 to 7 or peptide with an amino acid linker according to clause 14 or 15, wherein the linker moiety or the amino acid linker does not contain amino acids selected from the group consisting of proline, arginine, or histidine.
   17. Immunogenic compound or peptide with an amino acid linker according to clause 16, wherein no Y amino acid residue or YE dipeptide chain is present at the C-terminal end of the peptide, wherein Y is tyrosine and E is as defined above; preferably wherein
(a) X₁₂ is present and wherein no further amino acid residue is present C-terminally to X₁₂; or
(b) X₁₂ is not present and wherein no further amino acid residue is present C-terminally to X₁₁.
   18. Use of an immunogenic compound according to any one of clauses 1 to 7, 16 and 17 or an antigenic peptide according to clauses 12 or 13 or a peptide with an amino acid linker according to clauses 14 to 17 for the manufacture of a medicament for the treatment or prevention of synucleopathies, preferably for the treatment or prevention of a synucleinopathy selected from the group consisting of Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I).
   19. A method for the treatment or prevention of synucleopathies, preferably for the treatment or prevention of a synucleinopathy selected from the group consisting of Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I), wherein an effective amount of an immunogenic compound according to any one of clauses 1 to 7, 16 and 17 or an antigenic peptide according to clauses 12 or 13 or a peptide with an amino acid linker according to clauses 14 to 17, is administered to a human individual in need of such treatment or prevention.

### Abbreviations Used Herein

aa Amino acid
ab Antibody
aSyn Alpha synuclein
BSA Bovine serum albumin
bSyn Beta synuclein
CNS Central nervous system
DLB Dementia with Lewy bodies
DHA Docosahexaenoic acid (purified)
EC₅₀ Half-maximal effective concentration
ELISA Enzyme-linked immunosorbent assay
Fc Flow cell
FELASA Federation for laboratory animal science associations
h Hour(s)
HBS HEPES buffer saline
HNE 4-hydroxy-2-nonenal
HPLC High pressure liquid chromatography
HRP Horseradish peroxidase
IC₅₀ Half-maximal inhibitory concentration
IHC Immunohistochemistry
IQR Interquartile range
IR Immune response
kDa Kilodalton
KLH Keyhole limpet haemocyanin
LB Lewy body/Lewy bodies
mAb Monoclonal antibody
MSA Multiple system atrophy
OD Optical density
OD_{max/2} Half-maximal optical density
PD Parkinson's disease
RT Room temperature
RU Response units
SAIT Specific active immunotherapy
s.c. Subcutaneous
SEM Standard error of the mean
SN Substantia nigra
SPR Surface plasmon resonance
vs Versus
wt Wild type

The invention is further described by the following examples and the figures, yet without being limited thereto.

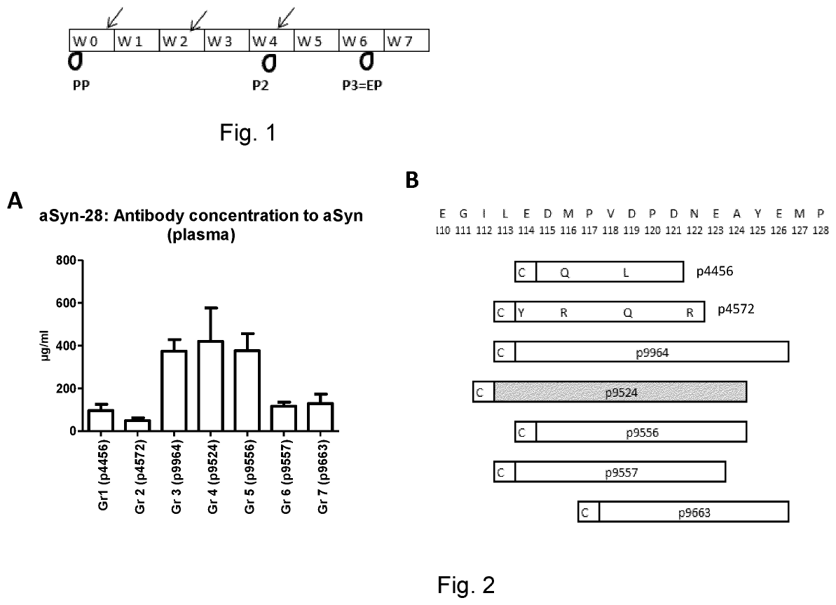

FIG. 1: Schematic time-course of the experiments. Injections are represented by arrows and blood sampling by drops. PP: Pre-plasma, EP: End-plasma. Pn: Plasma n. Wn: Week n of the experiment.

FIG. 2: Comparison of aSyn cross-reacting antibodies induced by p4456 and p4572 or by the corresponding native aSyn epitope of different length. (A) Induced antibody concentrations against aSyn protein from end-plasma of all individual mice are presented. Columns represent mean values with SEM. Extreme outliers (beyond 3× the IQR) have been removed from the graph (B) Relative position of the injected peptides along the amino-acid sequence of the native aSyn sequence. p9524 was used as scaffold for further development of AFFITOPE® s and is highlighted in gray.

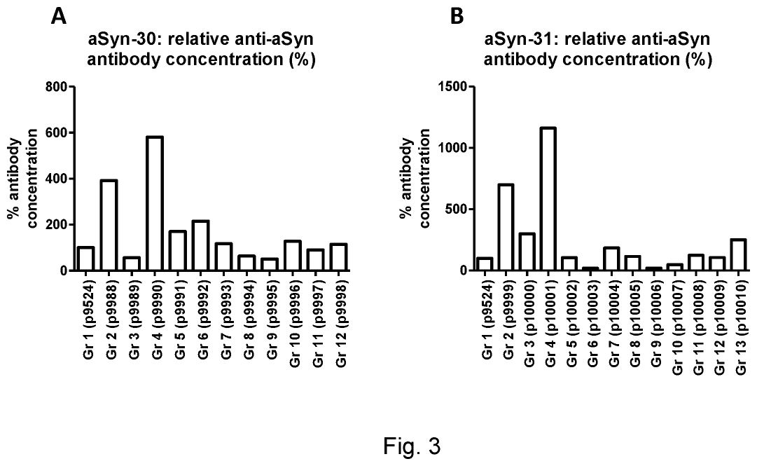

FIG. 3: Immunogenicity of peptides (delivered as peptide-carrier protein conjugates) with aSyn sequence aa113-124 with single alanine (A) or serine (B) exchanges. Group median concentrations of anti-aSyn antibodies in the end-plasma derived from individual immunized mice were determined and set in relation to the concentration of anti-aSyn antibodies elicited by vaccination with p9524, which was set as 100%. Sequences of the injected peptides are shown in Table 3 and 4.

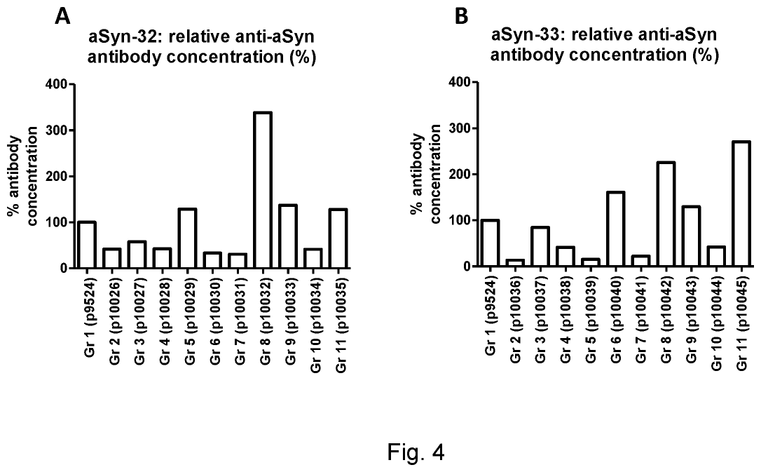

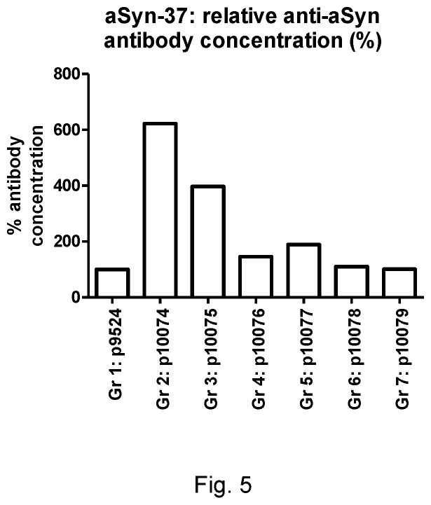

FIG. 4: Immunogenicity of peptides (delivered as peptide-carrier protein conjugates) with aSyn sequence aa113-124 with single amino acid exchanges at position 1 or 3 (A) and 4 or 12 (B). Group median concentrations of anti-aSyn antibodies in the end-plasma derived from individual immunized mice were determined and set in relation to the concentration of anti-aSyn antibodies elicited by vaccination with p9524, which was set as 100%. Sequences of the injected peptides are shown in Table 7 and 8.

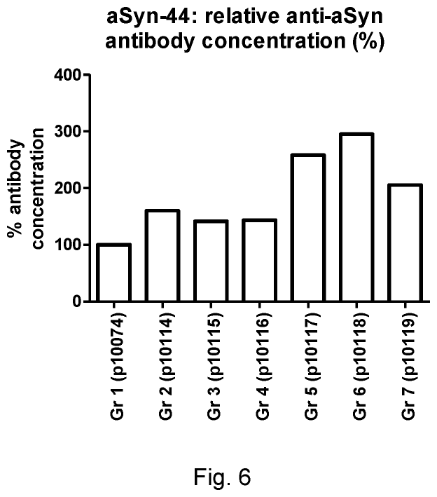

FIG. 5: Immunogenicity to aSyn induced by aSyn sequence aa₁₁₃₋₁₂₄ with double serine and other amino acid exchanges. Group median concentrations of anti-aSyn antibodies in the end-plasma derived from individual immunized mice were determined and set in relation to the concentration of anti-aSyn antibodies elicited by vaccination with p9524, which was set as 100%. Sequences of the injected peptides are shown in Table 11.

FIG. 6: Immunogenicity to aSyn induced by aSyn sequences aa₁₁₃₋₁₂₄ and N-terminally prolonged sequences. Group median concentrations of anti-aSyn antibodies in the end-plasma derived from individual immunized mice were determined and set in relation to the concentration of anti-aSyn antibodies elicited by vaccination with p10074, which was set as 100%. Sequences of the injected peptides are shown in Table 13.

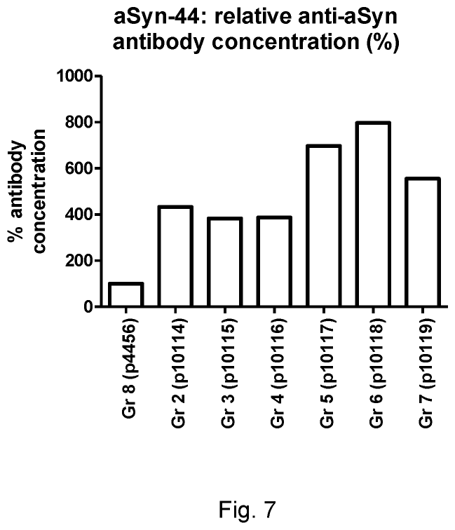

FIG. 7: Immunogenicity to aSyn induced by aSyn sequences aa₁₁₃₋₁₂₄, N-terminally prolonged sequences, and by the aSyn sequence aa₁₁₅₋₁₂₁ p4456. Group median concentrations of anti-aSyn antibodies in the end-plasma derived from individual immunized mice were determined and set in relation to the concentration of anti-aSyn antibodies elicited by vaccination with p4456, which was set as 100%. Sequences of the injected peptides are shown in Table 13.

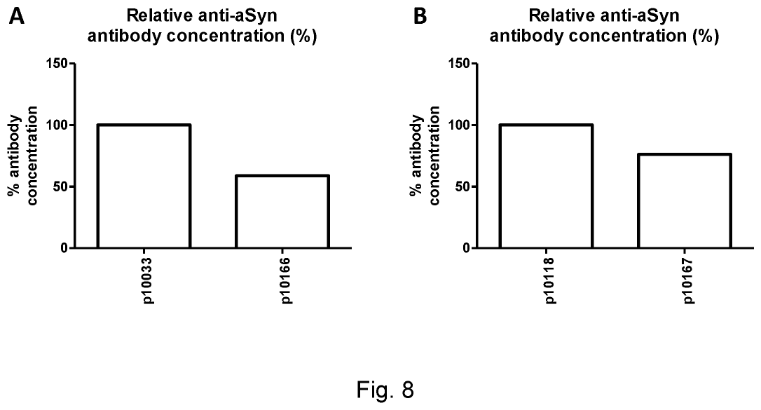

FIG. 8: Immunogenicity of aSyn targeting sequences p10033 and p10118 and C-terminally truncated sequences thereof. (A) Group median immunogenicity of anti-aSyn antibodies in the end-plasma derived from individual immunized mice were determined and set in relation to the concentration of anti-aSyn antibodies elicited by vaccination with p10033, which was set as 100%. (B) Group median immunogenicity of anti-aSyn antibodies in the end-plasma derived from individual immunized mice were determined and set in relation to the concentration of anti-aSyn antibodies elicited by vaccination with p10118, which was set as 100%. Sequences of the injected peptides are shown in Table 16.

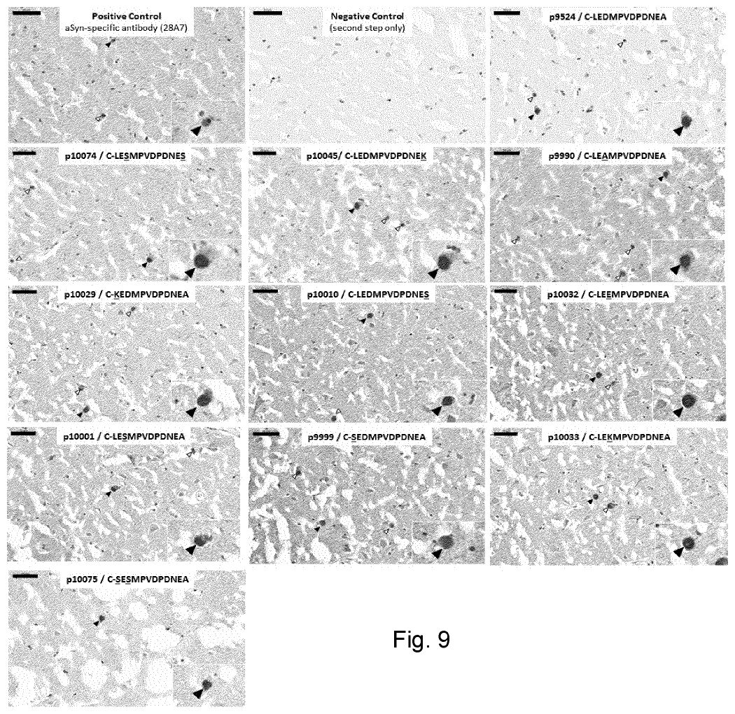

FIG. 9: IHC staining of post mortem human DLB brains by AFFITOPE® candidate-induced antibodies. The antibodies used in each panel are induced by the peptide shown on top of the respective panel. Underlined letters indicate amino acids that are different from the native sequence. Size bars indicate 50 μM in the main pictures and 10 μM in the smaller box in the lower right corner focusing on single Lewy Bodies (LBs).

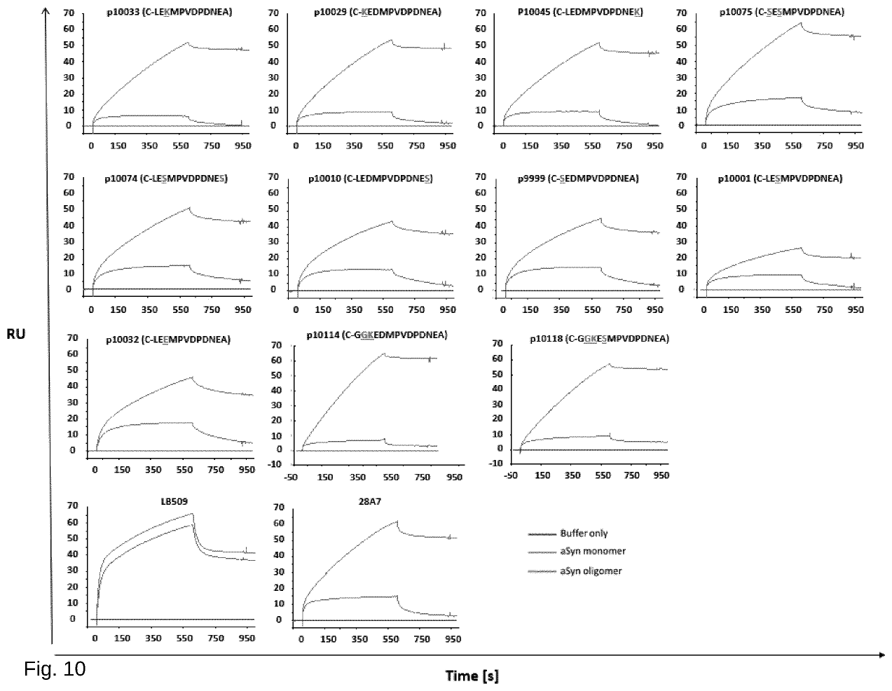

FIG. 10: Preferential binding to the oligomeric and toxic aSyn species vs the monomeric form (BiaCore data). Sensograms of the stability binding of AFFITOPE® candidate-induced antibodies and monoclonal antibodies LB509 and 28A7 to the oligomeric (red curve) or monomeric aSyn (green curve) species. The blue lines represent the negative control (binding to HBS buffer only). X-axis: running time (sec), y-axis: relative binding response units.

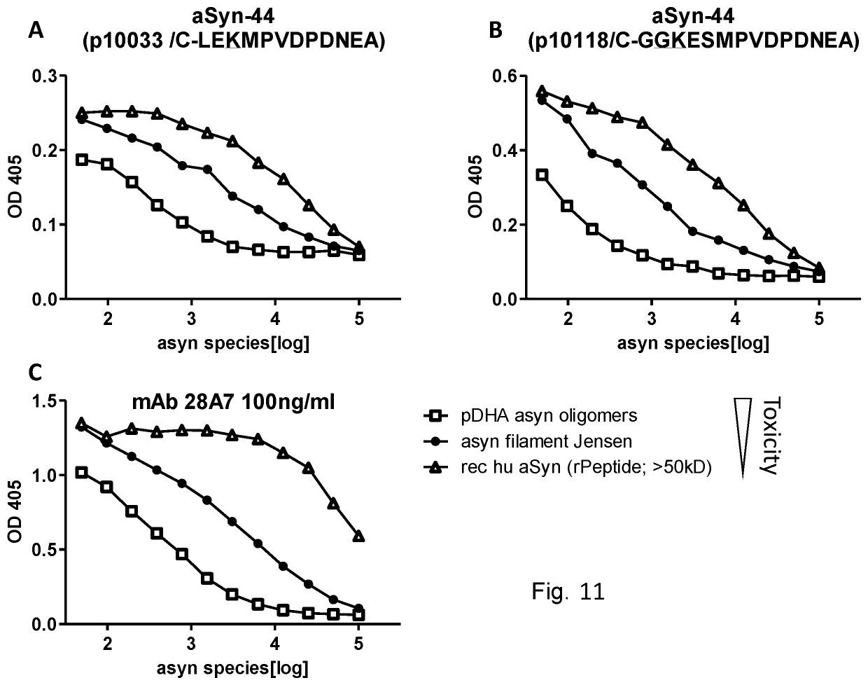

FIG. 11: Competition ELISA showing concentration-dependent inhibition of the binding of AFFITOPE® candidate-specific antibodies to aSyn monomers, oligomers, and filaments. Purified antibodies from AFFITOPE® candidate-immunized mice were pre-incubated with increasing amounts of different aSyn-species and then tested for binding to plate bound aSyn-oligomers. Inhibition of binding by monomeric aSyn is shown in red curves, inhibition by fibrillar aSyn in blue curves, and inhibition by oligomers in green curves. X-axis: decadic logarithm of aSyn-species concentration (ng/ml), y-axis: OD 405 value measured with each aSyn species and concentration.

### EXAMPLES

Identification of aSyn-AFFITOPE® s

Given the potential toxicity of various aSyn aggregates, therapeutic approaches for synucleinopathies might involve reducing the levels or accumulation of intracellular and extracellular aSyn. The fact that oligomeric aSyn is prone to be secreted into the intercellular space and is able to move from one affected neuron or (e.g. in the case of MSA) oligodendrocyte to a neigh-boring neuron or neuroglial cell in a prion-like fashion (Lee et al., 2008, Lashuel et al., 2013, Bengoa-Vergniory et al., 2017, Bernis et al., 2015) opens an avenue for a therapeutic approach such as AFFIRIS specific active immunotherapy (SAIT), that targets aSyn-transmission in a long-term manner. The AFFITOPE® s PD01 and PD03—short synthetic peptides mimicking defined regions of aSyn—have been tested as compounds for SAIT in clinical Phase I trials in PD—PD01A (NCT01568099; Volc et al., 2020) and PD03A (NCT02267434) —and in multiple system atrophy (MSA) patients (PD01 and PD03) (NCT02270489). The agents were shown to be well tolerated and elicited aSyn-specific antibodies with a preference for oligomeric aSyn.

In the course of the present invention a second-generation of aSyn-targeting AFFITOPE® s were developed, which induced higher titers and cross-reactivity to aSyn protein, than seen for PD01 and PD03, respectively. Furthermore, the focus of selection was also on the ability of the induced antibodies to discriminate between the aggregated and toxic aSyn species (oligomeric aSyn) and the monomeric aSyn protein. Thus, the binding of AFFITOPE®-induced antibodies to the overrepresented monomeric aSyn species in the periphery is supposed to be of minor extent, however, the binding to the toxic oligomeric and underrepresented aSyn species in the CNS and the periphery would occur preferably.

To identify highly potential AFFITOPE® s the aSyn epitope aa₁₁₃₋₁₂₄ was targeted. Well-defined selection criteria were applied such as high immunogenicity, high cross-reactivity to aSyn native epitope, and binding of induced antibodies with high selectivity towards oligomeric and fibrillar toxic aSyn species (oligomer binding>fibril binding).

The selection strategy consisted of several steps: (i) epitope finding within the C-terminus of aSyn target protein in order to identify a highly immunogenic and suitable epitope, (ii) alanine scan in order to detect positions along the native target sequence that can be exchanged in order to enhance immunogenicity and cross-reactivity, (iii) serine scans in order to detect positions along the native target sequence that can be exchanged in order to enhance immunogenicity and cross-reactivity, (iv) double serine exchanges along the native target sequence, (v) other than Ala or Ser amino acid exchanges able to improve immunogenicity and cross-reactivity towards aSyn, and (vi) removal of one amino acid at the C-terminus from two selected AFFITOPE® sequences.

### Immunization Schedule of all In Vivo Experiments

In all experiments described herein with BALB/c, mice received 3 injections with AFFITOPE® s or native aSyn epitope sequences (10 μg net peptide per injection), in biweekly intervals (FIG. 1).

Epitope Finding within the C-Terminus of the aSyn Target Protein (aSyn-28 In Vivo Experiment)

Epitope screening for highly immunogenic sequences along the C-terminal region of the aSyn protein has been performed. For this purpose, differently long amino acid stretches varying between 7 and 13 amino acids were used for immunogenicity studies in wt BALB/c mice (Table 1, FIG. 1). In parallel, previously selected AFFITOPE® s by AFFiRiS p4456 and p4572 were included for comparison. Plasmas from all individual animals were collected two weeks after the third immunization and subsequently analyzed by ELISA in order to determine the titers against the injected peptide and aSyn protein as well as the concentrations of aSyn reacting antibodies. The peptide p9524 corresponding to aSyn aa₁₁₃₋₁₂₄ sequence induced the highest amount of aSyn-specific antibodies (FIG. 2, Table 2). Based on these findings this sequence was selected as native target sequence for further AFFITOPE® candidate selection. The two other peptides p9964 and p9556 which elicited also high anti-aSyn titers were excluded from further development. The peptide p9964 includes the amino acids Y₁₂₅ and E₁₂₆ and peptide fragments derived from p9964 are predicted by in silico analyses to bind with high affinity to different allelic variants of MHCI and thus be potential cytotoxic T cell epitopes (www.syfpeithi.de). The peptide p9556 was not chosen for further development because it does not cover the potentially pathology-related calpain cleavage site aSyn L₁₁₃/E₁₁₄. Table 2 summarizes the titers to aSyn found in AFFITOPE® induced mouse plasmas.

Table 1: Setup of experiments aSyn-28. The table shows the drug product, the sequence of the peptide in the drug product and the corresponding sequence ID numbers.

TABLE 1

| Seq | | | | Additional |
|---|---|---|---|---|
| ID | Group | Drug Product | Sequence | information |
| 1 | 1 | p4456-CRM197 | C-DQPVLPD | aSyn115-121 (M→Q, |
| | | Aluminium hydroxide | | D→L) |
| 2 | 2 | p4572-CRM197 | C-YDRPVQPDR | aSyn114-122 (E→Y, |
| | | Aluminium hydroxide | | M→R, D→Q, N→R) |
| 3 | 3 | p9964-CRM197- | C-EDMPVDPDNEAYE | aSyn114-126 |
| | | Aluminium hydroxide | | |
| 4 | 4 | p9524-CRM197 | C-LEDMPVDPDNEA | aSyn113-124 |
| | | Aluminium hydroxide | | |
| 5 | 5 | p9556-CRM197 | C-DMPVDPDNEA | aSyn115-124 |
| | | Aluminium hydroxide | | |
| 6 | 6 | p9557-CRM197 | C-EDMPVDPDNE | aSyn114-123 |
| | | Aluminium hydroxide | | |
| 7 | 7 | p9663-CRM197 | C-VDPDNEAYE | aSyn118-126 |
| | | Aluminium hydroxide | | |

Table 2: aSyn-28, key results including titers, induced antibody concentration, and cross-reactivity. All parameters were evaluated in single mice and values refer to medians. The concentration of anti-aSyn antibodies in the plasma of immunized mice was extrapolated from a reference curve generated with the mAb LB509.

TABLE 2

| | | Titer against | Titer against | Ab conc. to |
|---|---|---|---|---|
| Seq ID | Group | injected peptide | aSyn | aSyn [μg/ml] |
| 1 | 1 | 98101 | 22951 | 97.0 |
| 2 | 2 | 114544 | 12449 | 49.5 |
| 3 | 3 | 62282 | 88308 | 375.1 |
| 4 | 4 | 90418 | 91136 | 420.9 |
| 5 | 5 | 78491 | 90089 | 376.9 |
| 6 | 6 | 33753 | 29295 | 117.5 |
| 7 | 7 | 93592 | 30084 | 129.7 |

Alanine-Scan Along the aSyn113-124 Epitope (aSyn-30) and Serine-Scan (aSyn-31)

As a next step, each amino acid position of the native aSyn₁₁₃₋₁₂₄ sequence was exchanged either by an alanine or a serine in order to identify substitutable positions in order to maintain or enhance immunogenicity (Tables 3 and 4). BALB/c mice were injected three times with the respective AFFITOPE® s and two weeks after the third and last injection, plasma of each individual mouse was collected and analyzed by ELISA in order to determine the AFFITOPE®-induced titers and aSyn antibody concentrations. An alanine scan revealed that the Leu at position 1 (p9988) and Asp at position 3 (p9990) along the aSyn 113-124aa (p9524) sequence are not essential, and on the contrary, their exchange to Ala leads to higher cross-reactivity to aSyn (FIG. 3A). The serine scan i.e. exchanges of each native occurring amino acids along the aSyn 113-124 sequence with serine (FIG. 3B) revealed a higher cross-reactivity to aSyn by amino acid exchanges at position 1 (p9999), 2 (p10000), 3 (p10001), and 12 (p10010) to Ser. Tables 5 and 6 summarize the induced titers relative to the titers induced by the WT sequence against aSyn present in the plasma of immunized mice.

Table 3: Setup of experiment aSyn-30. The table shows the different treatment groups, the respective drug product in use, the sequence of the peptide in the drug product, and the corresponding sequence ID numbers.

TABLE 3

| Seq | | | |
|---|---|---|---|
| ID | Group | Drug Product | Sequence |
| 4 | 1 | p9524-CRM197 Aluminum hydroxide | C-LEDMPVDPDNEA |
| 8 | 2 | p9988-CRM197 Aluminum hydroxide | C-AEDMPVDPDNEA |
| 9 | 3 | p9989-CRM197 Aluminum hydroxide | C-LADMPVDPDNEA |
| 10 | 4 | p9990-CRM197 Aluminum hydroxide | C-LEAMPVDPDNEA |
| 11 | 5 | p9991-CRM197 Aluminum hydroxide | C-LEDAPVDPDNEA |
| 12 | 6 | p9992-CRM197 Aluminum hydroxide | C-LEDMAVDPDNEA |
| 13 | 7 | p9993-CRM197 Aluminum hydroxide | C-LEDMPADPDNEA |
| 14 | 8 | p9994-CRM197 Aluminum hydroxide | C-LEDMPVAPDNEA |
| 15 | 9 | p9995-CRM197 Aluminum hydroxide | C-LEDMPVDADNEA |
| 16 | 10 | p9996-CRM197 Aluminum hydroxide | C-LEDMPVDPANEA |
| 17 | 11 | p9997-CRM197 Aluminum hydroxide | C-LEDMPVDPDAEA |
| 18 | 12 | p9998-CRM197 Aluminum hydroxide | C-LEDMPVDPDNAA |

Table 4: Setup of the experiment aSyn-31. The table shows the different treatment groups, the respective drug product in use, the sequence of the peptide in the drug product and the corresponding sequence ID numbers.

TABLE 4

| Seq | | | |
|---|---|---|---|
| ID | Group | Drug Product | Sequence |
| 4 | 1 | p9524-CRM197 Aluminum hydroxide | C-LEDMPVDPDNEA |
| 19 | 2 | p9999-CRM197 Aluminum hydroxide | C-SEDMPVDPDNEA |
| 20 | 3 | p10000-CRM197 Aluminum hydroxide | C-LSDMPVDPDNEA |
| 21 | 4 | p10001-CRM197 Aluminum hydroxide | C-LESMPVDPDNEA |
| 22 | 5 | p10002-CRM197 Aluminum hydroxide | C-LEDSPVDPDNEA |
| 23 | 6 | p10003-CRM197 Aluminum hydroxide | C-LEDMSVDPDNEA |
| 24 | 7 | p10004-CRM197 Aluminum hydroxide | C-LEDMPSDPDNEA |
| 25 | 8 | p10005-CRM197 Aluminum hydroxide | C-LEDMPVSPDNEA |
| 26 | 9 | p10006-CRM197 Aluminum hydroxide | C-LEDMPVDSDNEA |
| 27 | 10 | p10007-CRM197 Aluminum hydroxide | C-LEDMPVDPSNEA |
| 28 | 11 | p10008-CRM197 Aluminum hydroxide | C-LEDMPVDPDSEA |
| 29 | 12 | p10009-CRM197 Aluminum hydroxide | C-LEDMPVDPDNSA |
| 30 | 13 | p10010-CRM197 Aluminum hydroxide | C-LEDMPVDPDNES |

Table 5: aSyn-30, key results. The titers to aSyn evaluated in single mice and values represent medians relative to the median obtained with p9524.

TABLE 5

| Seq ID | Group | Titer relative to p9524 (%) |
|---|---|---|
| 4 | 1 | 100.0 |
| 8 | 2 | 391.8 |
| 9 | 3 | 56.2 |
| 10 | 4 | 581.0 |
| 11 | 5 | 170.0 |
| 12 | 6 | 215.5 |
| 13 | 7 | 117.0 |
| 14 | 8 | 63.9 |
| 15 | 9 | 50.1 |
| 16 | 10 | 127.7 |
| 17 | 11 | 90.4 |
| 18 | 12 | 114.4 |

Table 6: aSyn-31, key results. The titers to aSyn were evaluated in single mice and values represent medians relative to the median obtained with p9524.

TABLE 6

| Seq ID | Group | Titer relative to p9524 (%) |
|---|---|---|
| 4 | 1 | 100.0 |
| 19 | 2 | 699.1 |
| 20 | 3 | 300.5 |
| 21 | 4 | 1161.7 |
| 22 | 5 | 105.0 |
| 23 | 6 | 19.5 |
| 24 | 7 | 183.9 |
| 25 | 8 | 115.2 |
| 26 | 9 | 21.2 |
| 27 | 10 | 49.7 |
| 28 | 11 | 124.6 |
| 29 | 12 | 107.3 |
| 30 | 13 | 251.3 |

Amino Acid Exchanges on Positions 1, 3, 4, and 12 Along the aSyn aa113-124 Epitope Sequence (In Vivo Experiments aSyn-32 and aSyn-33)

Since exchanges of Leu at position 1, Asp at 3, Met at 4 and Ala at 12 into Ala and/or Ser of the native aSyn₁₁₃₋₁₂₄ sequence induced high aSyn specific antibody concentrations (FIG. 3), these positions are promising candidates for modifications leading to the design of AFFITOPES®. As a next step these positions were exchanged by amino acids with different properties (e.g. differently charged amino acids or amino acids with different polarity) in order to define favorable or less favorable exchanges with regard to immunogenicity and aSyn cross-reactivity (Tables 7 and 8). For comparison, a group injected with the native aSyn₁₁₃₋₁₂₄ (p9524) was included. The plasmas of immunized mice were analyzed by ELISA and antibody titers relative to the titers elicited with the native sequence against aSyn were determined (FIG. 4A, B). Exchanges to Lys at both positions 1 and 3 resulted in the induction of higher amounts of aSyn-specific Abs (p10029 and p10033), whereas exchanges to Trp dramatically reduced aSyn specific antibody production (p10026 and p10031) (FIG. 4A). In addition, the Asp to Glu exchange at position 3 resulted in higher cross-reactivity to aSyn in comparison to the native aSyn sequence (FIG. 4A). Met at position 4 and Ala at position 12 were exchanged with a similar strategy in experiment aSyn-33 (Table 8) as shown for position 1 and 3 in experiment aSyn-32. Here again, exchanges to Lys or Val at position 12 (p10045 or p10042) resulted in higher anti-aSyn antibody titers, whereas exchange to Trp at either position 4 or 12 reduced the cross-reactivity to aSyn (FIG. 4B). Tables 9 and 10 summarize the induced titers relative to the titers induced by the WT sequence against aSyn present in the plasma of immunized mice.

Table 7: Setup of experiment aSyn-32. The table shows the different treatment groups, the respective drug product in use, the sequence of the peptide in the drug product and the corresponding sequence ID numbers.

TABLE 7

| Seq | | | |
|---|---|---|---|
| ID | Group | Drug Product | Sequence |
| 4 | 1 | p9524-CRM197 Aluminum hydroxide | C-LEDMPVDPDNEA |
| 31 | 2 | p10026-CRM197 Aluminum hydroxide | C-WEDMPVDPDNEA |
| 32 | 3 | p10027-CRM197 Aluminum hydroxide | C-IEDMPVDPDNEA |
| 33 | 4 | p10028-CRM197 Aluminum hydroxide | C-NEDMPVDPDNEA |
| 34 | 5 | p10029-CRM197 Aluminum hydroxide | C-KEDMPVDPDNEA |
| 35 | 6 | p10030-CRM197 Aluminum hydroxide | C-DEDMPVDPDNEA |
| 36 | 7 | p10031-CRM197 Aluminum hydroxide | C-LEWMPVDPDNEA |
| 37 | 8 | p10032-CRM197 Aluminum hydroxide | C-LEEMPVDPDNEA |
| 38 | 9 | p10033-CRM197 Aluminum hydroxide | C-LEKMPVDPDNEA |
| 39 | 10 | p10034-CRM197 Aluminum hydroxide | C-LELMPVDPDNEA |
| 40 | 11 | p10035-CRM197 Aluminum hydroxide | C-LENMPVDPDNEA |

Table 8: Setup of experiment aSyn-33. The table shows the different treatment groups, the respective drug product in use, the sequence of the peptide in the drug product and the corresponding sequence ID numbers.

TABLE 8

| Seq | | | |
|---|---|---|---|
| ID | Group | Drug Product | Sequence |
| 4 | 1 | p9524-CRM197 Aluminum hydroxide | C-LEDMPVDPDNEA |
| 41 | 2 | p10036-CRM197 Aluminum hydroxide | C-LEDWPVDPDNEA |
| 42 | 3 | p10037-CRM197 Aluminum hydroxide | C-LEDLPVDPDNEA |
| 43 | 4 | p10038-CRM197 Aluminum hydroxide | C-LEDNPVDPDNEA |
| 44 | 5 | p10039-CRM197 Aluminum hydroxide | C-LEDDPVDPDNEA |
| 45 | 6 | p10040-CRM197 Aluminum hydroxide | C-LEDKPVDPDNEA |
| 46 | 7 | p10041-CRM197 Aluminum hydroxide | C-LEDMPVDPDNEW |
| 47 | 8 | p10042-CRM197 Aluminum hydroxide | C-LEDMPVDPDNEV |
| 48 | 9 | p10043-CRM197 Aluminum hydroxide | C-LEDMPVDPDNEG |
| 49 | 10 | p10044-CRM197 Aluminum hydroxide | C-LEDMPVDPDNED |
| 50 | 11 | p10045-CRM197 Aluminum hydroxide | C-LEDMPVDPDNEK |

Table 9: aSyn-32, key results. The titers to aSyn were evaluated in single mice and values represent medians relative to the median obtained with p9524.

TABLE 9

| Seq ID | Group | Titer relative to p9524 (%) |
|---|---|---|
| 4 | 1 | 100.0 |
| 31 | 2 | 41.9 |
| 32 | 3 | 57.7 |
| 33 | 4 | 42.5 |
| 34 | 5 | 128.9 |
| 35 | 6 | 33.1 |
| 36 | 7 | 30.9 |
| 37 | 8 | 338.4 |
| 38 | 9 | 137.2 |
| 39 | 10 | 41.4 |
| 40 | 11 | 128.2 |

Table 10: aSyn-33, key results. The titers to aSyn were evaluated in single mice and values represent medians relative to the median obtained with p9524.

TABLE 10

| Seq ID | Group | Titer relative to p9524 (%) |
|---|---|---|
| 4 | 1 | 100.0 |
| 41 | 2 | 13.6 |
| 42 | 3 | 84.6 |
| 43 | 4 | 41.5 |
| 44 | 5 | 15.2 |
| 45 | 6 | 160.8 |
| 46 | 7 | 22.2 |
| 47 | 8 | 225.5 |
| 48 | 9 | 129.5 |
| 49 | 10 | 42.2 |
| 50 | 11 | 270.8 |

Double Serine-Exchanges Along the aSyn aa113-124 Epitope Sequence (In Vivo Experiment aSyn-37)

VARIOTOPES® with additional serine exchanges along the aSyn₁₁₃₋₁₂₄ native sequence (p9524) were designed and tested for their immunogenicity in wt BALB/c mice (Table 11, FIG. 5). Two weeks after the third and last injection, plasma of each individual mouse was collected and analyzed by ELISA in order to determine the AFFITOPE®-induced titers and aSyn antibody concentrations. The native aSyn sequence was injected for direct comparison purposes (Group 1). Double Ser exchanges at positions 3 and 12 (p10074), as well as at positions 1 and 3 (p10075), were able to further enhance the titers of aSyn-specific antibody when compared to the native target sequence (FIG. 5). Double serine exchanges at position 1 and 4 as well as position 4 and 12 did not increase the immunogenicity towards aSyn (FIG. 5). Table 12 summarizes the induced titers relative to the titers induced by the WT sequence against aSyn present in the plasma of immunized mice.

Table 11: Setup of experiment aSyn-37. The table shows the different treatment groups, the respective drug product in use, the sequence of the peptide in the drug product and the corresponding sequence ID numbers.

TABLE 11

| Seq | | | |
|---|---|---|---|
| ID | Group | Drug Product | Sequence |
| 4 | 1 | p9524-CRM197 Aluminum hydroxide | C-LEDMPVDPDNEA |
| 51 | 2 | p10074-CRM197 Aluminum hydroxide | C-LESMPVDPDNES |
| 52 | 3 | p10075-CRM197 Aluminum hydroxide | C-SESMPVDPDNEA |
| 53 | 4 | p10076-CRM197 Aluminum hydroxide | C-LESSPVDPDNEA |
| 54 | 5 | p10077-CRM197 Aluminum hydroxide | C-SEDMPVDPDNES |
| 55 | 6 | p10078-CRM197 Aluminum hydroxide | C-LEDSPVDPDNES |
| 56 | 7 | p10079-CRM197 Aluminum hydroxide | C-SEDSPVDPDNEA |

Table 12: aSyn-37, key results. The titers to aSyn were evaluated in single mice and values represent medians relative to the median obtained with p9524.

TABLE 12

| Seq ID | Group | Titer relative to p9524 (%) |
|---|---|---|
| 4 | 1 | 100.0 |
| 51 | 2 | 622.3 |
| 52 | 3 | 397.0 |
| 53 | 4 | 145.9 |
| 54 | 5 | 188.8 |
| 55 | 6 | 110.5 |
| 56 | 7 | 101.3 |

Amino Acid Exchanges with Ser and Lys on Position 1, 3, and 12 Along the aSyn aa113-124 Epitope Sequence (In Vivo Experiment aSyn-44)

The exchanges to Ser and Lys at the positions 1, 3, and 12 of the native aSyn₁₁₃₋₁₂₄ sequence have been shown to be favorable to induce high anti-aSyn antibody concentrations (FIGS. 3B, 4 and 5). In the immunogenicity study presented herein combinations of serine and lysine exchanges along the aSyn₁₁₃₋₁₂₄ epitope were tested in wt BALB/c mice (aSyn-44 experiment, Table 13). In addition, linker amino acids have been added to different peptides. In parallel, the previously selected AFFITOPE® s candidate p4456 was included in this experiment for direct comparison. Two weeks after the third and last injection plasma of each individual mouse was collected and analyzed by ELISA in order to determine the AFFITOPE®-induced titers and aSyn antibody concentrations. The AFFITOPE® candidates tested in group 2-7 were able to induce higher titers of anti-aSyn antibodies when compared to the highly successful AFFITOPE® p10074 presented in FIG. 5 (FIG. 6). Especially, AFFITOPE® p10118 revealed to induce the highest titers against aSyn. Table 14 summarizes the induced titers relative to the titers induced by p10074 against aSyn present in the plasma of immunized mice.

In order to directly compare the immunogenicity of the newly selected AFFITOPE® sequences (group 2-7) to the peptide p4456 previously selected at AFFiRiS, the group median concentrations of anti-aSyn antibodies induced by these AFFITOPE® candidates were set in relation to the group median anti-aSyn antibody concentration elicited by p4456 (FIG. 7). All new AFFITOPE® candidates tested induced significantly higher aSyn specific titres compared to p4456 (FIG. 7). Table 15 summarizes the induced titers relative to the titers induced by the AFFITOPE® sequence p4456 against aSyn present in the plasma of immunized mice.

Table 13: Setup of experiment aSyn-44. The table shows the different treatment groups, the respective drug product in use, the sequence of the peptide in the drug product and the corresponding sequence ID numbers.

TABLE 13

| Seq ID | Group | Drug Product | Sequence |
|---|---|---|---|
| 51 | 1 | p10074-CRM197 Aluminum hydroxide | C-LESMPVDPDNES |
| 57 | 2 | p10114-CRM197 Aluminum hydroxide | CGG-KEDMPVDPDNEA |
| 58 | 3 | p10115-CRM197 Aluminum hydroxide | C-LEKMPVDPDNES |
| 59 | 4 | p10116-CRM197 Aluminum hydroxide | C-SEKMPVDPDNEA |
| 60 | 5 | p10117-CRM197 Aluminum hydroxide | C-LEKMPVDPDNEK |
| 61 | 6 | p10118-CRM197 Aluminum hydroxide | CGG-KESMPVDPDNEA |
| 62 | 7 | p10119-CRM197 Aluminum hydroxide | CGG-KESMPVDPDNEK |
| 1 | 8 | p4456-CRM197 Aluminum hydroxide | C-DQPVLPD |

Table 14: aSyn-44, key results. The titers to aSyn were evaluated in single mice and values represent medians relative to the median obtained with p10074 set to 100%.

TABLE 14

| Seq ID | Group | Titer relative to p10074 (%) |
|---|---|---|
| 51 | 1 | 100.0 |
| 57 | 2 | 160.1 |
| 58 | 3 | 141.6 |
| 59 | 4 | 143.4 |
| 60 | 5 | 258.2 |
| 61 | 6 | 295.1 |
| 62 | 7 | 205.5 |

Table 25: The titers to aSyn were evaluated in single mice and values represent group medians relative to the group median obtained with p4456 set to 100%.

TABLE 15

| Seq ID | Group | Titer relative to p4456 (%) |
|---|---|---|
| 1 | 8 | 100.0 |
| 57 | 2 | 432.5 |
| 58 | 3 | 382.5 |
| 59 | 4 | 387.3 |
| 60 | 5 | 697.5 |
| 61 | 6 | 797.1 |
| 62 | 7 | 555.2 |

C-Terminal Truncation of the Two Selected AFFITOPE® Sequences p10033 and p10118 by Removing the Ala at Position 12

AFFITOPE® sequences p10033 and especially p10118 have been shown to induce high anti-aSyn antibody concentrations (FIG. 4A and FIG. 6, respectively). In the immunogenicity studies presented herein the C-terminal Ala at position X12 was removed from AFFITOPE® sequences p10033 and p10118 in order to test whether C-terminal truncation has an influence on the immunogenicity and aSyn cross-reactivity of designed peptides. wt BALB/c mice where immunized in independent experiments with either p10033, p10118, p10166 or p10167. Table 16 lists the peptide sequences. Two weeks after the third injection plasma of each individual mouse was collected and analyzed by ELISA in order to determine the AFFITOPE®-induced titers and aSyn antibody reactivity. The truncated AFFITOPE® candidates tested were able to induce titers of anti-aSyn antibodies that, although lower than those generated using the AFFITOPE® sequences incorporating the Ala at position X₁₂ (FIG. 8), were still higher than those obtained using p4456. Table 17 summarizes the induced titers relative to the titers induced by either by the AFFITOPE® sequence p10033 (A) or by the AFFITOPE® sequence p10118 (B) against aSyn present in the plasma of immunized mice.

Table 16: The table shows the different treatment groups, the respective drug product in use, the sequence of the peptide in the drug product and the corresponding sequence ID numbers.

TABLE 16

| Seq ID | Drug Product | Sequence |
|---|---|---|
| 38 | p10033-CRM197 Aluminum hydroxide | C-LEKMPVDPDNEA |
| 63 | p10166-CRM197 Aluminum hydroxide | C-LEKMPVDPDNE |
| 61 | p10118-CRM197 Aluminum hydroxide | CGG-KESMPVDPDNEA |
| 64 | p10167-CRM197 Aluminum hydroxide | CGG-KESMPVDPDNE |

Table 17: The titers to aSyn were evaluated in single mice and values represent medians relative to the median obtained with either p10033 or p10118.

TABLE 17

| Seq ID | Titer relative to p10033 or p10118 (%) |
|---|---|
| 38 | 100.0 |
| 63 | 58.8 |
| 61 | 100.0 |
| 64 | 76.2 |

### Target Engagement of Antibodies Induced by Selected AFFITOPE® s

To investigate whether antibodies induced by AFFITOPE® candidate were able to detect aggregated aSyn in situ, sera of AFFITOPE® candidate-vaccinated wt BALB/c mice were tested by IHC staining on brain sections of human brain tissue derived postmortem of PD/DLB-diseased patients. Lewy bodies are a pathological hallmark in brains of PD/DLB-diseased patients and are mainly enriched with pathogenic, aggregated forms of aSyn (Spillantini et al., 1997).

Plasma from AFFITOPE® candidate-treated mice detected Lewy Bodies on brain sections of the cortex similar to the control anti-aSyn mAb 28A7 (FIG. 9). Specificity was confirmed by the lack of staining after pre-absorption of the serum with the corresponding peptide moiety of AFFITOPE® candidate (data not shown).

Preferential Binding to aSyn Oligomeric (Toxic) Vs Monomeric aSyn Species

Next, Abs induced by different AFFITOPE® s were tested for their selective binding to oligomeric aSyn (low MW, soluble aggregates of aSyn, predominantly di- and trimers) over the monomeric species using a SPR-based methodology (FIG. 10). Equal amounts of AFFITOPE® candidate-induced antibodies or monoclonal antibodies were first immobilized on an anti-mouse capture antibody coated chip. Subsequently monomeric and oligomeric aSyn species were applied consecutively, and the differential binding (defined as RU) to aSyn monomer and oligomeric aSyn species was assessed. As a control, two monoclonal antibodies were used: LB509 (Biolegend, San Diego, Calif.) and 28A7 (AFFiRiS AG). Immobilization of LB509 to the chip surface, which does not discriminate between monomeric and oligomeric aSyn, resulted in comparable RU to both aSyn species. The second control antibody, 28A7, which was raised against the peptide p4456, did discriminate between monomeric and oligomeric aSyn species. The AFFITOPE® candidate-induced antibodies showed high selectivity for oligomeric aggregates of aSyn compared to the monomeric form of aSyn (FIG. 10).

In addition to SPR (BiaCore) analyses, AFFITOPE®-induced Abs have been tested for their preferential binding to the aSyn filaments over the monomeric species of aSyn by inhibition ELISA. In these assays, a constant amount of affinity-purified AFFITOPE®-induced antibodies was preincubated with titrated amounts of monomeric and filamentous aSyn and then transferred to ELISA plates coated with aSyn filaments (for details see M&M). In FIG. 11, the results of two representative AFFITOPE® candidates (p10033 and p10118) are shown. Very good competition was seen with aSyn oligomers followed by aSyn filaments, whereas the competition with the monomeric form of aSyn was only of minor extent (FIG. 11).

A mAb 28A7 that was known to preferably bind the oligomeric and aggregated forms of aSyn was used as control (FIG. 11C).

Altogether, the binding data for AFFITOPE® candidate-specific antibodies provide clear evidence of high selectivity of AFFITOPE® candidate-induced antibodies to the toxic, oligomeric aggregates of aSyn, which are considered to be the relevant toxic species that lead to cell death, as opposed to the monomeric form.

### Material and Methods

### Mice

BALB/c mice were purchased from Janvier Elevages (Le Genest-Sainte-Isle, F).

The animals were housed and kept under standard conditions described in the application of the IMP to license its activity as breeders, suppliers and users. The respective permission was granted by the relevant authorities on May 13 2013 with the notification GZ:223633/2013/4.

Briefly, mice were kept in TECNIPLAST Sealsafe NextIVC Blue Line—Cages (Milano, IT) á five mice. Cages were equipped with enrichment in the form of nesting material and little plastic houses for hiding/playing purposes. The age of the mice at the beginning of the experiment was between 6 and 8 weeks. They were provided with standard diet and acidified water ad libitum and were kept under a 12 hour light/dark cycle.

All animal experiments were done according to Austrian and European law and were granted permission by the Vienna City Ad-ministration, municipal department 58.

### Peptides and Proteins

Peptides used for immunization were purchased from EMC micro-collections (Tübingen, Germany).

CRM197 was purchased from Pfenex (San Diego, Calif.).

### Manufacturing of Immunogenic Products

All immunogenic AFFITOPE®-based products used in the described experiments are conjugates of the synthetic AFFITOPE® peptides to the carrier protein CRM197. The conjugation is a directed procedure using the side chain amino groups of lysine residues in CRM197 and the free thiol group of the amino (N)-terminal cysteine in the peptide. For the activation of CRM197, the aqueous CRM197 solution is adjusted to 10 mM phosphate buffered saline (PBS) and is then gently shaken with the bifunctional linker 4-maleimidobutyric acid N-hydroxysuccinimide ester (GMBS). Subsequently, excess of unreacted GMBS is removed by either dialysis or ultrafiltration. The obtained activated CRM197 solution is subsequently incubated with AFFITOPE® peptides dissolved in phosphate buffer (pH 6.7). The free thiol group of the cysteine within the peptide reacts with the maleimido group forming the final AFFITOPE®-CRM197 product.

### Application of the Immunogen

Vaccines are brought to ambient temperature, vortexed and applied subcutaneously (s.c.) in the flank of mice (200 μl) with an insulin 20 syringe with a G30-gauge (Omnican® 50, B. Braun Melsungen AG, Melsungen, Germany). Immunization is repeated three times in biweekly intervals.

### Sample Collection

Blood was taken two weeks after each injection. Plasma was collected and stored at −20° C. until analysis. Blood collection and sacrifice of the animals was performed using FELASA-approved procedures.

### Monoclonal Antibodies

In this study, two monoclonal Abs were used as controls: LB509 (Biolegend, CA, US) and 28A7. LB509 is a commercially available purified anti-aSyn, 115-121 Ab. The mouse mAb 28A7 (IgG1) was generated in-house with mouse B cell hybridomas (Mandler et al., 2014) against the AFFITOPE® PD01 which mimics the aSyn₁₁₅₋₁₂₁ epitope.

### Sample Collection

Blood was taken approximately two weeks after each injection, at least one day before the next injection. Plasma was collected and stored at −20° C. until analysis. Blood collection and sacrifice of the animals was performed using FELASA-approved procedures.

### Titer Determination by ELISA

Titers against the immunizing peptides and against the recombinant human aSyn protein were analyzed. The presence of AFFIOTPE®-induced antibodies in plasma of immunized mice was determined by ELISA. 96-well plates (Nunc-Maxisorp) were coated with either recombinant human aSyn (1 μg/ml) or the injected peptide (BSA-conjugate; 1 μM). Titers were calculated as EC₅₀-values with PRISM® 5.04 (GraphPad Inc, San Diego, Calif.) by non-linear regression analysis (four-parameter logistic fit function).

### Immunohistochemistry (IHC)

Recognition of aSyn-positive inclusions (Lewy bodies) was performed on post-mortem brain sections from a frontal cortex biopsy of a DLB patient, (DLB patient, case No. X₅₆₃₁, Department of Neuroscience at UCSD, La Jolla, Calif.).

Plasma from AFFITOPE®-immunized mice was used to stain sections from frontal cortex brain biopsies from DLB patients. After tissue preparation (rehydration, deparaffinization, antigen retrieval, and blocking), sections were incubated with diluted mouse plasma for 2 h at RT or overnight at 4° C. Sections were incubated for 1 h at RT with undiluted Dako EnVision HRP labelled polymer (Agilent, Santa Clara, Calif.). For each IHC staining, counterstaining was done with Haematoxylin, and after this step, slides were de-hydrated and mounted in Entellan (Sigma-Aldrich). Slides were scanned in the brightfield mode using a Panoramic (Mirax) Scanner 150 (Carl Zeiss Microlmaging GmbH).

### Surface Plasmon Resonance (Biacore) Analysis

All experiments were run on a Biacore T200 (GE Healthcare, Chicago, Ill.) using Biacore T200 Control Software 2.0.1. A CM5 chip was immobilized with an antibody from a commercially available mouse antibody capture kit (GE-Healthcare) according to manufacturer's instructions by amine coupling on flow-cells (Fc) 1 and Fc2 of the chip. Fc1 served as reference flow cell. The immobilization levels of the anti-mouse antibody for both Fc were comparable and resulted in approximately 11000 RU for Fc1 and Fc2.

To reach comparable levels of the captured antibodies, the injection time for each specific antibody was tested before the actual run and injection time was adjusted accordingly. This resulted in comparable capture levels for all tested antibodies including the unspecific antibodies captured on Fc1 in each cycle.

The experimental setup for one cycle was as follows:
1. Capturing of unspecific antibody on reference Fc1
2. Capturing of aSyn-specific antibody on Fc2
3. Injection of sample (oligomeric aSyn, monomeric aSyn, buffer only) over Fc1 and Fc2
4. Regeneration of Fc1 and Fc 2 with low pH; only anti-mouse antibody remains on the chip
5. Chip surface ready for next cycle

Preparation of oligomeric and monomeric aSyn: aSyn oligomers (SynAging, Vandceuvre-les-Nancy, FR) were thawed and diluted to 5 μg/ml in HBS shortly before injection. To remove the high molecular weight fraction from bona fide monomeric aSyn (rPeptide), aSyn was freshly dissolved, diluted to 5 μg/ml in HBS, and cut-off centrifuged for 10 min at 14.000×g using 50 kDa cut-off columns (Amicon Ultra 0.5 ml). As controls, two aSyn-specific antibodies were used, LB509 (BioLegend, San Diego, Calif.) and 28A7 (AFFiRiS AG).

### Affinity Purification of AFFITOPE® Specific Antibodies Out of Immune Plasmas

Iodoacetyl magnetic beads (FG-106, Bioclone Inc., San Diego, Calif.) were coupled with the respective peptide (HPLC purified) for 1 h at RT and the remaining excess free sites were blocked with cysteine for an additional hour. After the blocking reaction, AFFITOPE®-coupled beads were incubated with 150 μl plasma of mice immunized with the corresponding AFFITOPE® candidate (for 2 h at RT). The AFFITOPE®-specific Abs were then eluted with Elution Buffer (Thermo Scientific). Afterwards the eluents were concentrated by Ultra Centrifugation (Millipore) tubes (30 kDa) to a volume of 150 μl (equal to the input volume).

### Competition ELISA

Titrated amounts of different aSyn species, monomer (rPeptide), and aggregated forms including fibrils (Proteos Inc), or oligomers (Crossbeta Bicosciences), in concentrations ranging from 100 to 0.05 μg/ml (this corresponds to a range of 69 to 0.034 μM relating to monomeric aSyn) were pre-incubated with antibodies that were previously purified from end-plasma from AFFITOPE® candidate-immunized mice, as well as the control anti-aSyn antibody 28A7. Added aSyn species compete for the binding to plate-coated aSyn fibrils (Proteos Inc). IC₅₀ values were calculated as the concentration of either monomeric, oligomeric, or fibrilic aSyn which was needed to quench half of the ELISA signal. IC₅₀ values were calculated with PRISM® 5.04 (GraphPad Inc, San Diego, Calif.) by non-linear regression analysis (four-parameter logistic fit function).

TABLE 18
AFFITOPERs and original sequence peptides tested

| | Internal | | |
|---|---|---|---|
| Sequence | peptide | | |
| ID no | name | Peptide sequence | Additional Information |
| 1 | p4456 | C-DQPVLPD | aSyn115-121 (M116→Q, D11→L) |
| 2 | p4572 | C-YDRPVQPDR | aSyn114-122 (E114→Y, M116→R, |
| | | | D119→Q, N122→R) |
| 3 | p9964 | C-EDMPVDPDNEAYE | aSyn114-126 |
| 4 | p9524 | C-LEDMPVDPDNEA | aSyn113-124 |
| 5 | p9556- | C-DMPVDPDNEA | aSyn115-124 |
| 6 | p9557 | C-EDMPVDPDNE | aSyn114-123 |
| 7 | p9663 | C-VDPDNEAYE | aSyn118-126 |
| 8 | p9988 | C-AEDMPVDPDNEA | aSyn113-124 (L113→A) |
| 9 | p9989 | C-LADMPVDPDNEA | aSyn113-124 (E114→A) |
| 10 | p9990 | C-LEAMPVDPDNEA | aSyn113-124 (D115→A) |
| 11 | p9991 | C-LEDAPVDPDNEA | aSyn113-124 (M116→A) |
| 12 | p9992 | C-LEDMAVDPDNEA | aSyn113-124 (P117→A) |
| 13 | p9993 | C-LEDMPADPDNEA | aSyn113-124 (V118→A) |
| 14 | p9994 | C-LEDMPVAPDNEA | aSyn113-124 (D119→A) |
| 15 | p9995 | C-LEDMPVDADNEA | aSyn113-124 (P120→A) |
| 16 | p9996 | C-LEDMPVDPANEA | aSyn113-124 (D121→A) |
| 17 | p9997 | C-LEDMPVDPDAEA | aSyn113-124 (N122→A) |
| 18 | p9998 | C-LEDMPVDPDNAA | aSyn113-124 (E123→A) |
| 19 | p9999 | C-SEDMPVDPDNEA | aSyn113-124 (L113→S) |
| 20 | p10000 | C-LSDMPVDPDNEA | aSyn113-124 (E114→S) |
| 21 | p10001 | C-LESMPVDPDNEA | aSyn113-124 (D115→S) |
| 22 | p10002 | C-LEDSPVDPDNEA | aSyn113-124 (M116→S) |
| 23 | p10003 | C-LEDMSVDPDNEA | aSyn113-124 (P117→S) |
| 24 | p10004 | C-LEDMPSDPDNEA | aSyn113-124 (V118→S) |
| 25 | p10005 | C-LEDMPVSPDNEA | aSyn113-124 (D119→S) |
| 26 | p10006 | C-LEDMPVDSDNEA | aSyn113-124 (P120→S) |
| 27 | p10007 | C-LEDMPVDPSNEA | aSyn113-124 (D121→S) |
| 28 | p10008 | C-LEDMPVDPDSEA | aSyn113-124 (N122→S) |
| 29 | p10009 | C-LEDMPVDPDNSA | aSyn113-124 (E123→S) |
| 30 | p10010 | C-LEDMPVDPDNES | aSyn113-124 (A124→S) |
| 31 | p10026 | C-WEDMPVDPDNEA | aSyn113-124 (L113→W) |
| 32 | p10027 | C-IEDMPVDPDNEA | aSyn113-124 (L113→I) |
| 33 | p10028 | C-NEDMPVDPDNEA | aSyn113-124 (L113→N) |
| 34 | p10029 | C-KEDMPVDPDNEA | aSyn113-124 (L113→K) |
| 35 | p10030 | C-DEDMPVDPDNEA | aSyn113-124 (L113→D) |
| 36 | p10031 | C-LEWMPVDPDNEA | aSyn113-124 (D115→W) |
| 37 | p10032 | C-LEEMPVDPDNEA | aSyn113-124 (D115→E) |
| 38 | p10033 | C-LEKMPVDPDNEA | aSyn113-124 (D115→K) |
| 39 | p10034 | C-LELMPVDPDNEA | aSyn113-124 (D115→L) |
| 40 | p10035 | C-LENMPVDPDNEA | aSyn113-124 (D115→N) |
| 41 | p10036 | C-LEDWPVDPDNEA | aSyn113-124 (M116→W) |
| 42 | p10037 | C-LEDLPVDPDNEA | aSyn113-124 (M116→L) |
| 43 | p10038 | C-LEDNPVDPDNEA | aSyn113-124 (M116→N) |
| 44 | p10039 | C-LEDDPVDPDNEA | aSyn113-124 (M116→D) |
| 45 | p10040 | C-LEDKPVDPDNEA | aSyn113-124 (M116→K) |
| 46 | p10041 | C-LEDMPVDPDNEW | aSyn113-124 (A124→W) |
| 47 | p10042 | C-LEDMPVDPDNEV | aSyn113-124 (A124→V) |
| 48 | p10043 | C-LEDMPVDPDNEG | aSyn113-124 (A124→G) |
| 49 | p10044 | C-LEDMPVDPDNED | aSyn113-124 (A124→D) |
| 50 | p10045 | C-LEDMPVDPDNEK | aSyn113-124 (A124→K) |
| 51 | p10074 | C-LESMPVDPDNES | aSyn113-124 (D115→S, A124→S) |
| 52 | p10075 | C-SESMPVDPDNEA | aSyn113-124 (L113→S, D115→S) |
| 53 | p10076 | C-LESSPVDPDNEA | aSyn113-124 (D115→S, M116→S) |
| 54 | p10077 | C-SEDMPVDPDNES | aSyn113-124 (L113→S, A124→S) |
| 55 | p10078 | C-LEDSPVDPDNES | aSyn113-124 (M116→S, A124→S) |
| 56 | p10079 | C-SEDSPVDPDNEA | aSyn113-124 (L113→S, M116→S) |
| 57 | p10114 | CGG-KEDMPVDPDNEA | aSyn111-124 (I112→G, D115→K) |
| 58 | p10115 | C-LEKMPVDPDNES | aSyn113-124 (D115→K, A124→S) |
| 59 | p10116 | C-SEKMPVDPDNEA | aSyn113-124 (L113→S, D115→K) |
| 60 | p10117 | C-LEKMPVDPDNEK | aSyn113-124 (D115→K, A124→K) |
| 61 | p10118 | CGG-KESMPVDPDNEA | aSyn111-124 (I112→G, D115→K, |
| | | | P117→S) |
| 62 | p10119 | CGG-KESMPVDPDNEK | aSyn111-124 (I112→G, D115→K, |
| | | | P117→S, A124→K) |
| 63 | p10166 | C-LEKMPVDPDNE | aSyn113-123 (D115→K) |
| 64 | p10167 | CGG-KESMPVDPDNE | aSyn111-123 (I112→G, D115→K, |
| | | | P117→S) |

### REFERENCES

Dufty et al., Am J Pathol. 2007 May; 170(5):1725-38. [PubMed: 17456777]
Bassil et al., Proc Natl Acad Sci USA. 2016 Aug. 23; 113(34): 9593-8. doi: 10.1073/pnas.1609291113 [PubMed: 27482103]
Kosaka, J Neurol. 1990; 237:197-204. [PubMed: 2196340]
Dickson et al., Acta Neuropathol. 1989; 78:572-584. [PubMed: 2683563]
Braak et al., Acta Neuropathol. 2007; 114:231-241. [PubMed: 17576580]
Lee et al., Int J Biochem Cell Biol. 2008; 40:1835-1849. [PubMed: 18291704]
Bengoa-Vergniory et al., Acta Neuropathol. 2017; 134(6):819-838. doi: 10.1007/s00401-017-1755-1. [PubMed: 28803412]
Bernis et al., Acta Neuropathol Commun. 2015 Nov. 26; 3:75. doi: 10.1186/s40478-015-0254-7. [PubMed: 26612754]
Mandler et al., Acta Neuropathol. 2014; 127(6):861-79. doi: 10.1007/s00401-014-1256-4. [PubMed: 24525765]
Spillantini et al., Nature. 1997 Aug. 28; 388(6645):839-40. (PubMed: 9278044]
Lashuel, et al., Nat. Rev. Neurosci. 14, 38-48. doi: 10.1038/nrn3406
Volc, et al., Lancet Neurol. 19, 591-600. doi: 10.1016/S1474-4422(20)30136-8

1. An antigenic peptide comprising, consisting essentially of or consisting of the structure:
X₁-X₂-X₃-X₄-X₅-X₆-X₇-P-X₉-X₁₀-X₁₁-X₁₂,
wherein:
P is proline;
X₁ is L, K, A or S, wherein L is leucine, K is lysine, A is alanine and S is serine;
X₂ is E or S, wherein E is glutamic acid and S is as defined above;
X₃ is D, E, K, N, A or S, wherein N is asparagine, D is aspartic acid and E, K, A and S are as defined above;
X₄ is M, A, S, L or K, wherein M is methionine and A, S, L and K are as defined above;
X₅ is P or A as defined above;
X₆ is V, A or S, wherein V is valine and A and S are as defined above;
X₇ is D or S as defined above;
X₉ is D or A as defined above;
X₁₀ is N, S or A, wherein N, S and A are as defined above;
X₁₁ is E, A or S, wherein E, A and S are as defined above;
X₁₂ is present or not and, if present, is A, K, V, S, or G wherein G is glycine and A, K, V and S are as defined above,
with the proviso that X₁-X₂-X₃-X₄-X₅-X₆-X₇-P-X₉-X₁₀-X₁₁-X₁₂ is not L-E-D-M-P-V-D-P-D-N-E-A,
and which comprises between 1 and 5 amino acid differences compared with the amino acid sequence L-E-D-M-P-V-D-P-D-N-E-A, and further wherein the peptide does not comprise the dipeptide Y-E immediately following X₁₂, wherein Y is tyrosine and E is as defined above.

2. The antigenic peptide of claim 1 which comprises between 1 and 4 amino acid differences compared with the amino acid sequence L-E-D-M-P-V-D-P-D-N-E-A, preferably between 1 and 3 amino acid differences and most preferably 2 amino acid differences.

3. The antigenic peptide of claim 1 or 2 which comprises amino acid differences compared with the amino acid sequence L-E-D-M-P-V-D-P-D-N-E-A at one or more positions selected from X₁, X₃, X₄ and X₁₂.

4. The antigenic peptide of any preceding claim comprising, consisting essentially of or consisting of the structure
Xₐ-X_{b}-X₁-X₂-X₃-X₄-X₅-X₆-X₇-P-X₉-X₁₀-X₁₁-X₁₂,
wherein:
Xₐ is present or not and, if present, is G, wherein G is as defined in claim 1;
X_{b} is G, wherein G is as defined above; and
X₁-X₁₂ are as defined in claim 1.

5. The antigenic peptide of any preceding claim, which is 11-20 amino acids in length, preferably 12-14 amino acids in length.

6. The antigenic peptide of any preceding claim, which further comprises a terminal cysteine residue, preferably an N-terminal cysteine residue.

7. The antigenic peptide of any preceding claim, wherein X₁ is L, S, A, or K, X₂ is E or S, X₃ is S, D, E, A, K or N, X₄ is M, X₅ is P; X₆ is V; X₇ is D; X₉ is D; X₁₀ is N, and/or X₁₂ is A, S, K or V, preferably wherein X₁ is L or K, X₂ is E, X₃ is S, D, E, K or A, X₄ is M, X₁₀ is N, and/or X₁₂ is A, S or K, especially wherein X₁ is L or K, X₃ is D, K or S and X₁₂ is A.

8. The antigenic peptide of any preceding claim which is selected from the group consisting of AEDMPVDPDNEA, KESMPVDPDNEA, LESMPVDPDNEA, LESMPVDPDNES, SEDMPVDPDNEA, SEKMPVDPDNEA LEEMPVDPDNEA, SESMPVDPDNEA, LEDMPVDPDNES, LEAMPVDPDNEA, LEDMPVDPDNEK, LEDMPVDPDNEV, LEKMPVDPDNEK, LSDMPVDPDNEA, LEKMPVDPDNEA, LEKMPVDPDNES, LENMPVDPDNEA, KESMPVDPDNEK and KEDMPVDPDNEA, preferably SEDMPVDPDNEA, SEKMPVDPDNEA, LEEMPVDPDNEA, LEKMPVDPDNEK, LESMPVDPDNEA, LESMPVDPDNES, KESMPVDPDNEA, KEDMPVDPDNEA, LEKMPVDPDNES, LEKMPVDPDNEA and LESMPVDPDNES, especially LEKMPVDPDNEA, KESMPVDPDNEK, KESMPVDPDNEA and KEDMPVDPDNEA.

9. The antigenic peptide of any preceding claim which comprises, consists essentially of or consists of the amino acid sequence KESMPVDPDNEA, GKESMPVDPDNEA, GGKESMPVDPDNEA or CGGKESMPVDPDNEA.

10. An immunogenic compound comprising the antigenic peptide of any preceding claim and a carrier comprising T-cell epitopes attached to the antigenic peptide.

11. The immunogenic compound of claim 10, wherein the carrier comprising T-cell epitopes is attached to the antigenic peptide via a linker.

12. The immunogenic compound of claim 10 or 11 wherein the carrier comprising T-cell epitopes is attached at the N terminal end of the antigenic peptide.

13. The immunogenic compound according to claim 12, wherein the carrier protein is selected from the group consisting of Keyhole Limpet Hemocyanin (KLH), tetanus toxoid, heat-labile enterotoxin (LT), cholera toxin (CT), diphtheria toxin (DT) and variants thereof, especially CRM197, tetanus toxoids (TT), mutant toxins, albumin-binding proteins, and bovine serum albumin, preferably CRM197.

14. A pharmaceutical preparation comprising an antigenic peptide according to any one of claims 1 to 9 or an immunogenic compound according to any one of claims 10 to 13 and a pharmaceutically acceptable excipient, preferably in the form of a vaccine composition.

15. The pharmaceutical preparation according to claim 14, further comprising an adjuvant.

16. The pharmaceutical preparation according to claim 15, wherein the adjuvant is selected from the group consisting of MF59 aluminium phosphate, calcium phosphate, cytokines (e.g., IL-2, IL-12, GM-CSF), saponins (e.g., QS21), MDP derivatives, CpG oligos, IC31, LPS, MPLA, polyphosphazenes, and aluminium hydroxide, or mixtures thereof; especially with aluminium hydroxide as adjuvant.

17. The pharmaceutical preparation according to claim 15 or 16, wherein the antigenic peptide is contained in an amount from 0.1 ng to 10 mg, preferably 10 ng to 1 mg, in particular 100 ng to 100 μg.

18. The pharmaceutical preparation according to any one of claims 14 to 17, wherein the preparation is formulated for parenteral administration, such as subcutaneous, intradermal, intravenous or intramuscular administration.

19. The antigenic peptide according to any one of claims 1 to 9, immunogenic compound according to any one of claims 10 to 13 or pharmaceutical preparation according to any one of claims 14 to 18, for use as a medicament.

20. The antigenic peptide according to any one of claims 1 to 9, immunogenic compound according to any one of claims 10 to 13 or pharmaceutical preparation according to any one of claims 14 to 18, for use as in the treatment or prevention of a synucleinopathy.

21. Use of the antigenic peptide according to any one of claims 1 to 9, immunogenic compound according to any one of claims 10 to 13 or pharmaceutical preparation according to any one of claims 14 to 18, for the manufacture of a medicament for the treatment or prevention of a synucleinopathy.

22. A method for the treatment or prevention of a synucleinopathy comprising administering an effective amount of the antigenic peptide according to any one of claims 1 to 9, immunogenic compound according to any one of claims 10 to 13 or pharmaceutical preparation according to any one of claims 14 to 18, to a subject in need thereof.

23. The use according to any one of claim 20 or 21 or method according to claim 22 wherein the synucleinopathy is a primary synucleinopathy or a concomitant pathology.

24. The use or method according to claim 23 wherein the primary synucleinopathy is selected from the group consisting of Parkinson's disease (sporadic, familial with alpha-synuclein mutations, familial with mutations other than alpha-synuclein, pure autonomic failure and Lewy body dysphagia), Lewy Body dementia (LBD; including dementia with Lewy bodies (DLB) (“pure” Lewy body dementia), Parkinson's disease dementia (PDD)), or Diffuse Lewy Body Disease, multiple system atrophy (Shy-Drager syndrome, striatonigral degeneration and olivopontocerebellar atrophy).

25. The use or method according to claim 23 wherein the concomitant pathology is selected from the group consisting of sporadic Alzheimer's disease, familial Alzheimer's disease with APP mutations, familial Alzheimer's disease with PS-1, PS-2 or other mutations, familial British dementia, inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectasia, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

26. The use according to any one of claim 20 or 21 or method according to claim 22 wherein the synucleinopathy is selected from the group consisting of Lewy Body Disorders (LBDs), especially Parkinson's Disease (PD), Parkinson's Disease with Dementia (PDD) and Dementia with Lewy Bodies (DLB), as well as Multiple System Atrophy (MSA) or Neurodegeneration with Brain Iron Accumulation type I (NBIA Type I).

27. The use or method according to any one of claims 21 to 26 wherein a human subject is treated.


SEQUENCE LISTING (165 sequences)

SEQ ID NO: 1 - protein, 8 aa, Artificial Sequence
  peptide
CDQPVLPD

SEQ ID NO: 2 - protein, 10 aa, Artificial Sequence
  peptide
CYDRPVQPDR

SEQ ID NO: 3 - protein, 14 aa, Artificial Sequence
  peptide
CEDMPVDPDNEAYE

SEQ ID NO: 4 - protein, 13 aa, Artificial Sequence
  peptide
CLEDMPVDPDNEA

SEQ ID NO: 5 - protein, 11 aa, Artificial Sequence
  peptide
CDMPVDPDNEA

SEQ ID NO: 6 - protein, 11 aa, Artificial Sequence
  peptide
CEDMPVDPDNE

SEQ ID NO: 7 - protein, 10 aa, Artificial Sequence
  peptide
CVDPDNEAYE

SEQ ID NO: 8 - protein, 13 aa, Artificial Sequence
  peptide
CAEDMPVDPDNEA

SEQ ID NO: 9 - protein, 13 aa, Artificial Sequence
  peptide
CLADMPVDPDNEA

SEQ ID NO: 10 - protein, 13 aa, Artificial Sequence
  peptide
CLEAMPVDPDNEA

SEQ ID NO: 11 - protein, 13 aa, Artificial Sequence
  peptide
CLEDAPVDPDNEA

SEQ ID NO: 12 - protein, 13 aa, Artificial Sequence
  peptide
CLEDMAVDPDNEA

SEQ ID NO: 13 - protein, 13 aa, Artificial Sequence
  peptide
CLEDMPADPDNEA

SEQ ID NO: 14 - protein, 13 aa, Artificial Sequence
  peptide
CLEDMPVAPDNEA

SEQ ID NO: 15 - protein, 13 aa, Artificial Sequence
  peptide
CLEDMPVDADNEA

SEQ ID NO: 16 - protein, 13 aa, Artificial Sequence
  peptide
CLEDMPVDPANEA

SEQ ID NO: 17 - protein, 13 aa, Artificial Sequence
  peptide
CLEDMPVDPDAEA

SEQ ID NO: 18 - protein, 13 aa, Artificial Sequence
  peptide
CLEDMPVDPDNAA

SEQ ID NO: 19 - protein, 13 aa, Artificial Sequence
  peptide
CSEDMPVDPDNEA

SEQ ID NO: 20 - protein, 13 aa, Artificial Sequence
  peptide
CLSDMPVDPDNEA

SEQ ID NO: 21 - protein, 13 aa, Artificial Sequence
  peptide
CLESMPVDPDNEA

SEQ ID NO: 22 - protein, 13 aa, Artificial Sequence
  peptide
CLEDSPVDPDNEA

SEQ ID NO: 23 - protein, 13 aa, Artificial Sequence
  peptide
CLEDMSVDPDNEA

SEQ ID NO: 24 - protein, 13 aa, Artificial Sequence
  peptide
CLEDMPSDPDNEA

SEQ ID NO: 25 - protein, 13 aa, Artificial Sequence
  peptide
CLEDMPVSPDNEA

SEQ ID NO: 26 - protein, 13 aa, Artificial Sequence
  peptide
CLEDMPVDSDNEA

SEQ ID NO: 27 - protein, 13 aa, Artificial Sequence
  peptide
CLEDMPVDPSNEA

SEQ ID NO: 28 - protein, 13 aa, Artificial Sequence
  peptide
CLEDMPVDPDSEA

SEQ ID NO: 29 - protein, 13 aa, Artificial Sequence
  peptide
CLEDMPVDPDNSA

SEQ ID NO: 30 - protein, 13 aa, Artificial Sequence
  peptide
CLEDMPVDPDNES

SEQ ID NO: 31 - protein, 13 aa, Artificial Sequence
  peptide
CWEDMPVDPDNEA

SEQ ID NO: 32 - protein, 13 aa, Artificial Sequence
  peptide
CIEDMPVDPDNEA

SEQ ID NO: 33 - protein, 13 aa, Artificial Sequence
  peptide
CNEDMPVDPDNEA

SEQ ID NO: 34 - protein, 13 aa, Artificial Sequence
  peptide
CKEDMPVDPDNEA

SEQ ID NO: 35 - protein, 13 aa, Artificial Sequence
  peptide
CDEDMPVDPDNEA

SEQ ID NO: 36 - protein, 13 aa, Artificial Sequence
  peptide
CLEWMPVDPDNEA

SEQ ID NO: 37 - protein, 13 aa, Artificial Sequence
  peptide
CLEEMPVDPDNEA

SEQ ID NO: 38 - protein, 13 aa, Artificial Sequence
  peptide
CLEKMPVDPDNEA

SEQ ID NO: 39 - protein, 13 aa, Artificial Sequence
  peptide
CLELMPVDPDNEA

SEQ ID NO: 40 - protein, 13 aa, Artificial Sequence
  peptide
CLENMPVDPDNEA

SEQ ID NO: 41 - protein, 13 aa, Artificial Sequence
  peptide
CLEDWPVDPDNEA

SEQ ID NO: 42 - protein, 13 aa, Artificial Sequence
  peptide
CLEDLPVDPDNEA

SEQ ID NO: 43 - protein, 13 aa, Artificial Sequence
  peptide
CLEDNPVDPDNEA

SEQ ID NO: 44 - protein, 13 aa, Artificial Sequence
  peptide
CLEDDPVDPDNEA

SEQ ID NO: 45 - protein, 13 aa, Artificial Sequence
  peptide
CLEDKPVDPDNEA

SEQ ID NO: 46 - protein, 13 aa, Artificial Sequence
  peptide
CLEDMPVDPDNEW

SEQ ID NO: 47 - protein, 13 aa, Artificial Sequence
  peptide
CLEDMPVDPDNEV

SEQ ID NO: 48 - protein, 13 aa, Artificial Sequence
  peptide
CLEDMPVDPDNEG

SEQ ID NO: 49 - protein, 13 aa, Artificial Sequence
  peptide
CLEDMPVDPDNED

SEQ ID NO: 50 - protein, 13 aa, Artificial Sequence
  peptide
CLEDMPVDPDNEK

SEQ ID NO: 51 - protein, 13 aa, Artificial Sequence
  peptide
CLESMPVDPDNES

SEQ ID NO: 52 - protein, 13 aa, Artificial Sequence
  peptide
CSESMPVDPDNEA

SEQ ID NO: 53 - protein, 13 aa, Artificial Sequence
  peptide
CLESSPVDPDNEA

SEQ ID NO: 54 - protein, 13 aa, Artificial Sequence
  peptide
CSEDMPVDPDNES

SEQ ID NO: 55 - protein, 13 aa, Artificial Sequence
  peptide
CLEDSPVDPDNES

SEQ ID NO: 56 - protein, 13 aa, Artificial Sequence
  peptide
CSEDSPVDPDNEA

SEQ ID NO: 57 - protein, 15 aa, Artificial Sequence
  peptide
CGGKEDMPVDPDNEA

SEQ ID NO: 58 - protein, 13 aa, Artificial Sequence
  peptide
CLEKMPVDPDNES

SEQ ID NO: 59 - protein, 13 aa, Artificial Sequence
  peptide
CSEKMPVDPDNEA

SEQ ID NO: 60 - protein, 13 aa, Artificial Sequence
  peptide
CLEKMPVDPDNEK

SEQ ID NO: 61 - protein, 15 aa, Artificial Sequence
  peptide
CGGKESMPVDPDNEA

SEQ ID NO: 62 - protein, 15 aa, Artificial Sequence
  peptide
CGGKESMPVDPDNEK

SEQ ID NO: 63 - protein, 12 aa, Artificial Sequence
  peptide; XXXXPVDPDXEX is not LEDMPVDPDNEA
  VARIANT 1: can be L, K, A or S
  VARIANT 2: can be E or S
  VARIANT 3: can be D, E, K, N, A or S
  VARIANT 4: can be M, A, S, L or K
  VARIANT 10: can be N, S or A
  VARIANT 12: can be present or not, and if present, can be A, K, V, S or G
XXXXPVDPDXEX

SEQ ID NO: 64 - protein, 12 aa, Artificial Sequence
  peptide
KESMPVDPDNEA

SEQ ID NO: 65 - protein, 12 aa, Artificial Sequence
  peptide
LESMPVDPDNEA

SEQ ID NO: 66 - protein, 12 aa, Artificial Sequence
  peptide
LESMPVDPDNES

SEQ ID NO: 67 - protein, 12 aa, Artificial Sequence
  peptide
SEDMPVDPDNEA

SEQ ID NO: 68 - protein, 12 aa, Artificial Sequence
  peptide
LEEMPVDPDNEA

SEQ ID NO: 69 - protein, 12 aa, Artificial Sequence
  peptide
SESMPVDPDNEA

SEQ ID NO: 70 - protein, 12 aa, Artificial Sequence
  peptide
LEDMPVDPDNES

SEQ ID NO: 71 - protein, 12 aa, Artificial Sequence
  peptide
LEAMPVDPDNEA

SEQ ID NO: 72 - protein, 12 aa, Artificial Sequence
  peptide
LEDMPVDPDNEK

SEQ ID NO: 73 - protein, 12 aa, Artificial Sequence
  peptide
LEDMPVDPDNEV

SEQ ID NO: 74 - protein, 12 aa, Artificial Sequence
  peptide
LEKMPVDPDNEK

SEQ ID NO: 75 - protein, 12 aa, Artificial Sequence
  peptide
LSDMPVDPDNEA

SEQ ID NO: 76 - protein, 12 aa, Artificial Sequence
  peptide
LEKMPVDPDNEA

SEQ ID NO: 77 - protein, 12 aa, Artificial Sequence
  peptide
KEDMPVDPDNEA

SEQ ID NO: 78 - protein, 12 aa, Artificial Sequence
  peptide
LENMPVDPDNEA

SEQ ID NO: 79 - protein, 12 aa, Artificial Sequence
  peptide
KESMPVDPDNEK

SEQ ID NO: 80 - protein, 12 aa, Artificial Sequence
  peptide
KEDMPVDPDNEA

SEQ ID NO: 81 - protein, 12 aa, Artificial Sequence
  peptide
SEDMPVDPDNEA

SEQ ID NO: 82 - protein, 12 aa, Artificial Sequence
  peptide
LEEMPVDPDNEA

SEQ ID NO: 83 - protein, 12 aa, Artificial Sequence
  peptide
LESMPVDPDNEA

SEQ ID NO: 84 - protein, 12 aa, Artificial Sequence
  peptide
KESMPVDPDNEA

SEQ ID NO: 85 - protein, 12 aa, Artificial Sequence
  peptide
KEDMPVDPDNEA

SEQ ID NO: 86 - protein, 12 aa, Artificial Sequence
  peptide
LEKMPVDPDNEA

SEQ ID NO: 87 - protein, 12 aa, Artificial Sequence
  peptide
LESMPVDPDNES

SEQ ID NO: 88 - protein, 12 aa, Artificial Sequence
  peptide
LEKMPVDPDNEA

SEQ ID NO: 89 - protein, 12 aa, Artificial Sequence
  peptide
KESMPVDPDNEA

SEQ ID NO: 90 - protein, 12 aa, Artificial Sequence
  peptide
KEDMPVDPDNEA

SEQ ID NO: 91 - protein, 12 aa, Artificial Sequence
  peptide
KESMPVDPDNEA

SEQ ID NO: 92 - protein, 12 aa, Artificial Sequence
  peptide
KESMPVDPDNEA

SEQ ID NO: 93 - protein, 12 aa, Artificial Sequence
  peptide
LESMPVDPDNEA

SEQ ID NO: 94 - protein, 12 aa, Artificial Sequence
  peptide
LESMPVDPDNES

SEQ ID NO: 95 - protein, 12 aa, Artificial Sequence
  peptide
SEDMPVDPDNEA

SEQ ID NO: 96 - protein, 12 aa, Artificial Sequence
  peptide
LEEMPVDPDNEA

SEQ ID NO: 97 - protein, 12 aa, Artificial Sequence
  peptide
SESMPVDPDNEA

SEQ ID NO: 98 - protein, 12 aa, Artificial Sequence
  peptide
LEDMPVDPDNES

SEQ ID NO: 99 - protein, 12 aa, Artificial Sequence
  peptide
LEAMPVDPDNEA

SEQ ID NO: 100 - protein, 12 aa, Artificial Sequence
  peptide
LEDMPVDPDNEK

SEQ ID NO: 101 - protein, 12 aa, Artificial Sequence
  peptide
LEDMPVDPDNEV

SEQ ID NO: 102 - protein, 12 aa, Artificial Sequence
  peptide
LEKMPVDPDNEK

SEQ ID NO: 103 - protein, 12 aa, Artificial Sequence
  peptide
LSDMPVDPDNEA

SEQ ID NO: 104 - protein, 12 aa, Artificial Sequence
  peptide
LEKMPVDPDNEA

SEQ ID NO: 105 - protein, 12 aa, Artificial Sequence
  peptide
KEDMPVDPDNEA

SEQ ID NO: 106 - protein, 12 aa, Artificial Sequence
  peptide
LENMPVDPDNEA

SEQ ID NO: 107 - protein, 12 aa, Artificial Sequence
  peptide
KESMPVDPDNEK

SEQ ID NO: 108 - protein, 12 aa, Artificial Sequence
  peptide
KEDMPVDPDNEA

SEQ ID NO: 109 - protein, 12 aa, Artificial Sequence
  peptide
SEDMPVDPDNEA

SEQ ID NO: 110 - protein, 12 aa, Artificial Sequence
  peptide
LEEMPVDPDNEA

SEQ ID NO: 111 - protein, 12 aa, Artificial Sequence
  peptide
LESMPVDPDNEA

SEQ ID NO: 112 - protein, 12 aa, Artificial Sequence
  peptide
KESMPVDPDNEA

SEQ ID NO: 113 - protein, 12 aa, Artificial Sequence
  peptide
KEDMPVDPDNEA

SEQ ID NO: 114 - protein, 12 aa, Artificial Sequence
  peptide
LEKMPVDPDNEA

SEQ ID NO: 115 - protein, 12 aa, Artificial Sequence
  peptide
LESMPVDPDNES

SEQ ID NO: 116 - protein, 12 aa, Artificial Sequence
  peptide
LEKMPVDPDNEA

SEQ ID NO: 117 - protein, 12 aa, Artificial Sequence
  peptide
KESMPVDPDNEA

SEQ ID NO: 118 - protein, 12 aa, Artificial Sequence
  peptide
KEDMPVDPDNEA

SEQ ID NO: 119 - protein, 14 aa, Artificial Sequence
  peptide
GGKESMPVDPDNEA

SEQ ID NO: 120 - protein, 13 aa, Artificial Sequence
  peptide
GKESMPVDPDNEA

SEQ ID NO: 121 - protein, 15 aa, Artificial Sequence
  peptide
GGGKESMPVDPDNEA

SEQ ID NO: 122 - protein, 15 aa, Artificial Sequence
  peptide
CGGKESMPVDPDNEA

SEQ ID NO: 123 - protein, 15 aa, Artificial Sequence
  peptide
GCGKESMPVDPDNEA

SEQ ID NO: 124 - protein, 15 aa, Artificial Sequence
  peptide
GGCKESMPVDPDNEA

SEQ ID NO: 125 - protein, 15 aa, Artificial Sequence
  peptide
CCGKESMPVDPDNEA

SEQ ID NO: 126 - protein, 15 aa, Artificial Sequence
  peptide
CGCKESMPVDPDNEA

SEQ ID NO: 127 - protein, 15 aa, Artificial Sequence
  peptide
CCCKESMPVDPDNEA

SEQ ID NO: 128 - protein, 14 aa, Artificial Sequence
  peptide
CCKESMPVDPDNEA

SEQ ID NO: 129 - protein, 14 aa, Artificial Sequence
  peptide
CGKESMPVDPDNEA

SEQ ID NO: 130 - protein, 14 aa, Artificial Sequence
  peptide
GCKESMPVDPDNEA

SEQ ID NO: 131 - protein, 13 aa, Artificial Sequence
  peptide
CKESMPVDPDNEA

SEQ ID NO: 132 - protein, 14 aa, Artificial Sequence
  peptide
GGKESMPVDPDNEK

SEQ ID NO: 133 - protein, 14 aa, Artificial Sequence
  peptide
GGKEDMPVDPDNEA

SEQ ID NO: 134 - protein, 14 aa, Artificial Sequence
  peptide
GGKESMPVDPDNEK

SEQ ID NO: 135 - protein, 14 aa, Artificial Sequence
  peptide
GGKEDMPVDPDNEA

SEQ ID NO: 136 - protein, 13 aa, Artificial Sequence
  peptide
CLESMPVDPDNEA

SEQ ID NO: 137 - protein, 13 aa, Artificial Sequence
  peptide
CLESMPVDPDNES

SEQ ID NO: 138 - protein, 13 aa, Artificial Sequence
  peptide
CSEDMPVDPDNEA

SEQ ID NO: 139 - protein, 13 aa, Artificial Sequence
  peptide
CLEEMPVDPDNEA

SEQ ID NO: 140 - protein, 13 aa, Artificial Sequence
  peptide
CSESMPVDPDNEA

SEQ ID NO: 141 - protein, 13 aa, Artificial Sequence
  peptide
CLEDMPVDPDNES

SEQ ID NO: 142 - protein, 13 aa, Artificial Sequence
  peptide
CLEAMPVDPDNEA

SEQ ID NO: 143 - protein, 13 aa, Artificial Sequence
  peptide
CLEDMPVDPDNEK

SEQ ID NO: 144 - protein, 13 aa, Artificial Sequence
  peptide
CLEDMPVDPDNEV

SEQ ID NO: 145 - protein, 15 aa, Artificial Sequence
  peptide
CGGKESMPVDPDNEA

SEQ ID NO: 146 - protein, 13 aa, Artificial Sequence
  peptide
CLEKMPVDPDNEK

SEQ ID NO: 147 - protein, 13 aa, Artificial Sequence
  peptide
CLSDMPVDPDNEA

SEQ ID NO: 148 - protein, 13 aa, Artificial Sequence
  peptide
CLEKMPVDPDNEA

SEQ ID NO: 149 - protein, 13 aa, Artificial Sequence
  peptide
CLEKMPVDPDNEA

SEQ ID NO: 150 - protein, 13 aa, Artificial Sequence
  peptide
CLENMPVDPDNEA

SEQ ID NO: 151 - protein, 15 aa, Artificial Sequence
  peptide
CGGKESMPVDPDNEK

SEQ ID NO: 152 - protein, 15 aa, Artificial Sequence
  peptide
CGGKEDMPVDPDNEA

SEQ ID NO: 153 - protein, 14 aa, Artificial Sequence
  peptide
GGKESMPVDPDNEA

SEQ ID NO: 154 - protein, 13 aa, Artificial Sequence
  peptide
GKESMPVDPDNEA

SEQ ID NO: 155 - protein, 15 aa, Artificial Sequence
  peptide
GGGKESMPVDPDNEA

SEQ ID NO: 156 - protein, 15 aa, Artificial Sequence
  peptide
CGGKESMPVDPDNEA

SEQ ID NO: 157 - protein, 15 aa, Artificial Sequence
  peptide
GCGKESMPVDPDNEA

SEQ ID NO: 158 - protein, 15 aa, Artificial Sequence
  peptide
GGCKESMPVDPDNEA

SEQ ID NO: 159 - protein, 15 aa, Artificial Sequence
  peptide
CCGKESMPVDPDNEA

SEQ ID NO: 160 - protein, 15 aa, Artificial Sequence
  peptide
CGCKESMPVDPDNEA

SEQ ID NO: 161 - protein, 15 aa, Artificial Sequence
  peptide
CCCKESMPVDPDNEA

SEQ ID NO: 162 - protein, 14 aa, Artificial Sequence
  peptide
CCKESMPVDPDNEA

SEQ ID NO: 163 - protein, 14 aa, Artificial Sequence
  peptide
CGKESMPVDPDNEA

SEQ ID NO: 164 - protein, 14 aa, Artificial Sequence
  peptide
GCKESMPVDPDNEA

SEQ ID NO: 165 - protein, 13 aa, Artificial Sequence
  peptide
CKESMPVDPDNEA